(19) United States
(12) Patent Application Publication
(10) Pub. No.: US 2023/0277474 A1
(43) Pub. Date: Sep. 7, 2023

(54) Storage Stable Films Comprising Fibrin and/or Fibrinogen

(71) Applicants: Vitruvian Medical Devices, Inc., San Francisco, CA (US); Octapharma AG, Lachen (CH)

(72) Inventors: Hobart W. Harris, San Francisco, CA (US); Daniel Grant Ericson, Rochester, MN (US)

(21) Appl. No.: 18/023,668

(22) PCT Filed: Aug. 31, 2021
(86) PCT No.: PCT/US2021/048401

§ 371 Date: Feb. 27, 2023

Related U.S. Application Data
(60) Provisional application No. 63/072,753, filed on Aug. 31, 2020.

(51) Int. Cl. A61K 9/70; A61K 38/36; A61K 33/38; A61K 35/16; A61P 17/02

(52) U.S. Cl. CPC A61K 9/7007; A61K 38/363; A61K 38/36; A61K 33/38; A61K 35/16; A61P 17/02

(57) ABSTRACT

Described herein are dried films comprising fibrin and/or fibrinogen, and methods of using same. In certain aspects, the dried films are storage stable at room temperature. In certain aspects, the dried films described herein are effective for preventing or treating a wound in a subject in need thereof.

### CROSS REFERENCE TO RELATED APPLICATIONS

This application claims the right of priority based on U.S. application Ser. No. 63/072,753, filed Aug. 31, 2020, which is herein incorporated in its entirety by reference.

### BACKGROUND

Application of fibrinogen or fibrin gels or films to wounds can enhance healing of the wound and prevent common complications. Incisional hernias, for example, are a frequent complication of abdominal surgery, resulting in considerable patient morbidity and increased health care costs. There are 4-5 million abdominal incisions (laparotomies) performed annually in the United States with hernias resulting after 11-23% of these procedures. Incisional hernias may result in severe morbidity beyond the cosmetic deformity of a visible bulge in the anterior abdominal wall, including intestinal obstruction, bowel ischemia, enterocutaneous fistula and significant limitations on a patient's physical activity and gainful employment. Consequently, there are over 400,000 incisional hernia repairs performed each year making it one of the most common procedures performed by general surgeons. The increase in U.S. health care costs due to incisional hernia repair is estimated to currently exceed eight billion dollars per year, not including the costs of unemployment benefits for this moderately young patient population. Research indicates that incisional hernias result from inadequate or impaired healing of the myofascial abdominal wall following surgery. Accordingly, each of the recognized risk factors for hernia formation inhibits wound healing, including morbid obesity, diabetes, smoking, chronic lung disease, surgical site infection and poor surgical technique. Since the incidence of the major risk factors is increasing, the prevalence of incisional hernias is predicted to increase as well.

Current compositions that are available for enhancing wound healing, such as surgical wounds and preventing incisional hernias, are time consuming and/or difficult to prepare, and lack significant storage stability for preparation prior to use. Novel methods and compositions for enhancing wound healing are needed that are easy to prepare and allow for rapid application, particularly in clinical settings.

Thin, dried, fibrin films polymerized with thrombin and/or calcium are described in WO2017210267. However, these films do not describe clinically relevant amounts of silver or preferred physical forms of silver, i.e., silver microparticles, to enhance wound healing properties of thin, dried fibrin films.

Identifying a delivery system for the silver microparticles and fibrin that is easy to use, economically viable, stoichiometrically accurate and consistent has historically proved very problematic. The current disclosure provides a solution to these problems. Fibrin is currently marketed as a kit containing fibrinogen and thrombin supplied either frozen or as a lyophilized powder, and thus needs to be thawed or reconstituted before use. Silver microparticles, supplied as a fine powder, do not distribute evenly into either product component due to their high density. Earlier products comprising liquid fibrin, thrombin, and silver are described in WO2013138238. In those products, the three components were manually mixed in the wound. This delivery method is acceptable for experimentation purposes, but inconvenient for wide-spread use and practically, untenable as an FDA-approved, commercial product.

The present disclosure provides a solution for these critical problems by providing a dry, thin film that contains a therapeutic mixture of silver and fibrin, is stable at extreme temperatures for several years, is easy to handle and apply, the silver is abrasion resistant, and can be readily integrated into current wound care workflows.

### SUMMARY

In one aspect, provided herein are dried films, comprising: fibrin, fibrinogen, or combinations thereof and silver microparticles, wherein the film contains about 0.01 International Unit (IU) or less of thrombin per cm² of film; and wherein the film is stable at room temperature for at least 18 months.

In some embodiments, the silver microparticles are present at a concentration of 1-50 mg silver/cm² of film.

In some embodiments, the silver microparticles are present at a concentration of 2.5-25 mg silver microparticles/cm² of film.

In some embodiments, the silver microparticles have a mean diameter ranging from about 2 μm to 1,000 μm.

In some embodiments, the silver microparticles have a mean diameter of about 15 μm.

In some embodiments, the fibrin or fibrinogen is present as a component of whole blood or whole plasma.

In some embodiments, the fibrin, fibrinogen or combinations thereof are present at an amount of 0.5 to 20.0 mg/cm² of film.

In some embodiments, the fibrinogen is present at an amount of 2.5 to 4.0 mg/cm² of film.

In some embodiments, the film does not contain thrombin.

In some embodiments, the film releases between 0.02 and 5.0 ppm silver ions when applied to a subject.

In some embodiments, the film has a moisture content of between about 0.5 to 2.0 mg/cm²

In some embodiments, the film has a moisture content of about 0.9 mg/cm².

In some embodiments, the film comprises silver microparticles on the surface of the film.

In some embodiments, the silver microparticles on the surface of the film are abrasion resistant.

In some embodiments, abrasion resistance is determined by brushing.

In some embodiments, the silver microparticles on the surface of the film are abrasion resistant to at least 100 brushing assay repeats.

In some embodiments, the film has a fold number of at least 2, at least 3, at least 4, or at least 5.

In some embodiments, the fold number is determined by folding the film until the film breaks or ruptures.

In some embodiments, the film has a fold endurance of at least 5, 10, 20, 30, 40, 50, 60, 70, 80, 90, 100, 500, or 1000.

In some embodiments, the fold endurance is determined by folding and unfolding the film on the same crease line until the film breaks or ruptures.

In some embodiments, the film has a burst pressure of about 50 to 1000 mm.

In some embodiments, the film has a burst pressure of at least 800 mm Hg.

In some embodiments, the film is stable at room temperature for at least 15 months.

In some embodiments, the stability is determined by burst pressure and fold endurance.

In some embodiments, the film has a fold number of at least 5 after storage at room temperature for 15 months.

In some embodiments, the film has a fold endurance of at least 100 after storage at room temperature for 15 months.

In some embodiments, the film has a burst pressure of at least 800 after storage at room temperature for 15 months.

In some embodiments, the film further comprises an adhesive coating.

In some embodiments, the adhesive coating is oxidized regenerated cellulose.

In some embodiments, the adhesive coating is present on the film at an amount of 5%-25% by weight of film.

In some embodiments, the film is sterile.

In some embodiments, the film is sterilized by gamma irradiation.

In some embodiments, the film further comprises calcium.

In some embodiments, the calcium is present in the film at a concentration of 0.00005-0.20 mg/cm² of film.

In some embodiments, the film has a thickness of between 0.1 mm and 1 mm.

In some embodiments, the film has a thickness of about 100 μm, 150 μm, or 200 μm.

In another aspect, provided herein are methods of treating a wound in a subject, comprising application of an effective amount of the dried film to the wound.

In some embodiments, the dried film is applied to the wound less than 15 minutes after opening a storage container comprising the dried film.

In some embodiments, the dried film is applied to the wound between 1 and 15 minutes after opening the storage container comprising the dried film.

In some embodiments, the wound is an acute wound, a chronic wound, a non-healing wound, a diabetic foot ulcer, a venous leg ulcer, a pressure ulcer, a surgical wound, an arterial wound, a traumatic wound, or a skin disorder defined by ICD-9 code.

In some embodiments, the wound is a non-healing wound.

In some embodiments, the wound is selected from the group consisting of decreased primary post-surgical adhesion formation, decreased post-surgical adhesion reformation, a wound from cellular and tissue engraftment, and a burn wound.

In some embodiments, the dried film degrades in less than 28 days after the application of the film to the wound of the subject.

In another aspect, provided herein are method of manufacturing a dried film for treating wounds in a subject, comprising: obtaining human plasma; placing the plasma into a mold; polymerizing the plasma to form a gel by adding a polymerizing agent to the plasma; adding silver microparticles to the gel to generate a silver microparticle-containing plasma gel; removing fluid from the polymerized gel to generate a film; drying the film; and gamma sterilizing the film.

In some embodiments, the removal of fluid to generate a film is performed by applying vacuum pressure to the gel at a pressure of about 1.25 Torr to 125 Torr.

In some embodiments, the polymerizing agent is CaCl₂ or thrombin.

In some embodiments, the polymerizing agent is CaCl₂.

In some embodiments, between 0.5-1 mg/ml CaCl₂ is added to the plasma.

In some embodiments, about 0.86 mg/ml CaCl₂ is added to the plasma.

In some embodiments, the method further comprises incubating the film with a glycerin solution prior to drying the film.

In some embodiments, the glycerin solution comprises 2% glycerin.

In some embodiments, the silver microparticles are added to the plasma.

In some embodiments, the silver microparticles are added before polymerization of the gel.

In some embodiments, the silver microparticles are added by mixing the silver microparticles with the plasma.

In some embodiments, the silver microparticles are added after polymerization of the gel.

In some embodiments, the silver microparticles are added before removal of the fluid from the gel.

In some embodiments, the silver microparticles are added after removal of the fluid from the gel.

In some embodiments, the silver microparticles are added after the glycerin solution incubation.

In some embodiments, the silver microparticles are added by applying a spray coating comprising silver microparticles on the polymerized gel.

In some embodiments, the silver microparticles are present at a concentration of 1-50 mg silver/cm² of film.

In some embodiments, the silver microparticles are present at a concentration of 2.5-25 mg silver/cm² of film.

In some embodiments, the silver microparticles have a mean diameter ranging from 2 μm to 1,000 μm.

In some embodiments, the silver microparticles have a mean diameter of about 15 μm.

In some embodiments, the film further comprises a phospholipid.

In some embodiments, the phospholipid is phosphatidylserine or phosphatidylcholine.

The method of any one of claims 44-65, wherein the calcium is present in the film at a concentration of 0.00005-0.20 mg/cm² of film.

In some embodiments, the film comprises less than about 0.0999 IU thrombin per cm² of film.

In some embodiments, the method further comprises incubating the plasma at 37° C. after placing the plasma into the mold.

In some embodiments, the plasma is incubated at 37° C. for 5 to 30 min.

In some embodiments, the plasma is incubated at 37° C. for 15 min.

In some embodiments, the method further comprises incubating the gel at 37° C. after incubation with the glycerin solution.

In some embodiments, the gel is incubated at 37° C. for 5 to 60 min.

In some embodiments, the gel is incubated at 37° C. for 5 min.

In some embodiments, the film is dried at a temperature of 42° C. to 50° C.

In some embodiments, the film is dried for 30 min to 120 min.

In some embodiments, the film is dried in a convection oven.

In some embodiments, the film has a moisture content of between about 0.5 to 2.0 mg/cm².

In some embodiments, the film has a moisture content of about 0.9 mg/cm².

In some embodiments, the method further comprises adding an adhesive coating to the film.

In some embodiments, the adhesive coating is oxidized regenerated cellulose.

In some embodiments, the adhesive coating is present on the film at an amount of 5%-25% by weight.

In some embodiments, the method further comprises virally inactivating the human plasma.

In some embodiments, the viral inactivation comprises solvent/detergent and/or nanofiltration.

In some embodiments, the solvent/detergent is Triton X-100.

In some embodiments, the film has a thickness of about between 0.1 mm and 1 mm.

In another aspect, provided herein are methods of preventing a wound in a subject, comprising application of an effective amount of the dried film to an area that is susceptible to forming a wound in the subject.

In some embodiments, the dried film is applied to the area that is susceptible to forming a wound less than 15 minutes after opening a storage container comprising the dried film.

In some embodiments, the dried film is applied to the area that is susceptible to forming a wound between 1 and 15 minutes after opening the storage container comprising the dried film.

In some embodiments, the wound is a non-healing wound.

In some embodiments, the wound is an acute wound, a chronic wound, a non-healing wound, a diabetic foot ulcer, a venous leg ulcer, a pressure ulcer, a surgical wound, an arterial wound, a traumatic wound, or a skin disorder defined by ICD-9 code.

In some embodiments, the wound is a non-healing wound.

In some embodiments, the wound is a hernia.

In some embodiments, the dried film degrades in less than 28 days after the application of the film to the area susceptible to forming a wound in the subject.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a diagram illustrating an embodiment of the dried films disclosed herein.

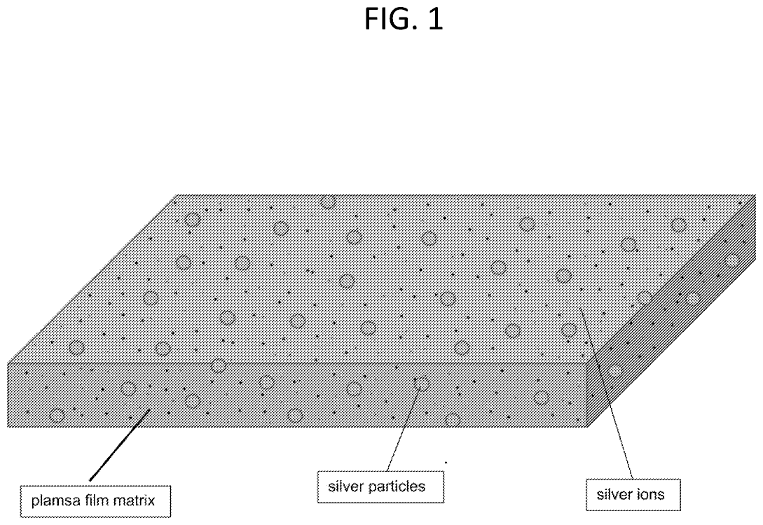

FIG. 2A-2D show silver microparticle ionization in various solutions. The microparticles were extracted in four liquid vehicles to four different time/temperature endpoints (1 h, 24 h, and 7 days at 37° C. and 72 h at 50° C.). Data are means±standard deviation (SD). FIG. 2A shows ionization in water. FIG. 2B shows ionization in simulated body fluid, SBF. FIG. 2C shows ionization in normal saline. FIG. 2D shows ionization in human plasma.

FIG. 3A shows that silver microparticles mixed within a plasma film accelerate cutaneous wound healing in genetically diabetic (db/db) mice. The mean area under the curve (AUC) analysis was significantly lower for the silver microparticles containing plasma film —treated animals versus saline-treated controls as determined by Student's t-test. FIG. 3B shows that silver microparticles plus a liquid fibrin sealant accelerate cutaneous wound healing in genetically diabetic (db/db) mice. The mean area under the curve (AUC) analysis was significantly lower for the silver microparticles plus a liquid fibrin sealant-treated animals versus saline-treated controls as determined by Student's t-test.

FIG. 4 shows a representative histology sample of healing excisional skin wound in a diabetic mouse (C57BL/KsJ-db/db) treated with the dried fibrin film containing silver microparticles L. Several multi-nucleated giant cells can be seen surrounding silver microparticles (black arrows) and surrounding air spaces resulting from dislodgement of silver microparticles during tissue processing (white arrows). 40× magnification.

FIG. 5 provides a schedule of events for a proposed clinical trial.

### DETAILED DESCRIPTION

### Definitions

Terms used in the claims and specification are defined as set forth below unless otherwise specified.

The term “about” means, except where explicitly stated otherwise, +/−15% of a specified value. In some embodiments, “about” means+/−5% of a specified value.

The term “dried film” as used herein refers to the films comprising fibrin/fibrinogen described herein (e.g., films comprising whole blood or whole plasma), wherein the film has been subjected to a process (e.g., application of pressure and/or heat) to substantially remove water from the film.

The term stability or “storage stability” refers to the extent to which the product retains, within specified limits and throughout its period of storage and use, the same properties and characteristics that it possessed at the time of its manufacture. For example, the product's stability can be measured as the film product having the same burst pressure that it possessed at the time of its manufacture, i.e., 800 to 1,300 mm Hg. An exemplary stability specific limit is maintenance of at least 80% of a quantitative metric after a selected amount of time as compared to a newly synthesized film. Quantitative metrics include, but are not limited to, burst pressure, fold number, and fold endurance. In some embodiments, stability is the combination of two or more quantitative metrics.

The term “enhanced wound healing” refers to an increase in the efficiency of wound healing and/or an increase in the strength of the resulting healed wound site as compared to a wound that has not been treated with the methods and compositions of the present disclosure. In certain instances, enhanced wound healing refers to increased deposition of mature collagen on a wound compared to a corresponding wound that has not been treated with the methods and compositions of the present disclosure.

The term “non-healing wounds” refers to wounds that do not heal without medical intervention or wounds that do not heal in a sufficient time without medical intervention that would likely lead to increased risk of infection or wound severity over time. Examples of non-healing wounds include, but are not limited to: diabetic foot ulcers, venous ulcers, pressure ulcers, arterial ulcers, chronic ulcers, traumatic wounds, surgical wounds and skin disorders.

The term “in situ” as used herein, refers to application of a pharmaceutical composition at on the subject (e.g., at the site of the wound of a subject).

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to enhance wound healing in a subject.

The term “therapeutically effective amount” is an amount that is effective to ameliorate a symptom of a disease

It must be noted that, as used in the specification and the appended claims, the singular forms “a,” “an” and “the” include plural referents unless the context clearly dictates otherwise.

Briefly, and as described in more detail below, described herein are methods and compositions for enhancing wound healing in a subject. Several features of the current approach should be noted. The composition is stable at room temperature of a minimum of three years and can be applied to the wound readily. The composition is applied as a dry film that allows for even application of the composition to the wound surface, including accurate dosing of the components therein. The compositions with fibrinogen/fibrin plus silver enhance wound healing, including the increased fusion of monocytes/macrophages into multinucleated giant cells and the deposition of mature collagen throughout the wound.

Films Comprising Fibrin/Fibrinogen and Silver

Described herein are dried films comprising fibrin, fibrinogen, or combinations thereof. In certain aspects, the dried films are storage stable at room temperature. In certain embodiments, the dried films are stable at room temperature for at least 3 years.

The fibrin and/or fibrinogen can be supplied as a component of whole blood, whole plasma, cryoprecipitate, or fibrinogen concentrate. The fibrinogen may also be derived from recombinant fibrinogen. Thus, in certain embodiments, the fibrin or fibrinogen is derived from whole plasma. In certain embodiments, the fibrin or fibrinogen is derived from a recombinant fibrinogen. In certain embodiments, the fibrin or fibrinogen is derived from a cryoprecipitated fibrin or fibrinogen.

In certain embodiments, the fibrin and/or fibrinogen is at a concentration of 0.5 to 20.0 mg/cm² film. In certain embodiments, the fibrin and/or fibrinogen is at a concentration of 1.0 to 10.0 mg/cm² film, 0.5 to 1.0 mg/cm² film, 0.5 to 5.0 mg/cm² film, 1.0 to 2.0 mg/cm² film, 2.0 to 3.0 mg/cm² film, 3.0 to 4.0 mg/cm² film, 4.0 to 5.0 mg/cm² film, 5.0 to 6.0 mg/cm² film, 6.0 to 7.0 mg/cm² film, 7.0 to 8.0 mg/cm² film, 8.0 to 9.0 mg/cm² film, 9.0 to 10.0 mg/cm² film, 1.5 to 2.0 mg/cm² film, 2.0 to 2.5 mg/cm² film, 2.5 to 3.0 mg/cm² film, 3.0 to 3.5 mg/cm² film, 3.5 to 4.0 mg/cm² film, 4.0 to 4.5 mg/cm² film, 4.5 to 5.0 mg/cm² film, 5.0 to 5.5 mg/cm² film, 5.5 to 6.0 mg/cm² film, 6.0 to 6.5 mg/cm² film, or, 6.5 to 7.0 mg/cm² film, 7.0 to 7.5 mg/cm² film, 7.5 to 8.0 mg/cm² film, 8.0 to 8.5 mg/cm² film, 8.5 to 9.0 mg/cm² film, 9.0 to 9.5 mg/cm² film, or 9.5 to 10.0 mg/cm² film. In certain embodiments, the fibrin and/or fibrinogen is at a concentration of about 4.0 mg/cm² film.

In certain embodiments, for every 1 mL plasma, there is 1-5 mg fibrinogen in the film. In certain embodiments, for every 1 mL plasma, there is 2-5 mg, 2.5-4.5 mg, 3.0-5.0 mg, 2.0-3.0 mg, 3.0-4.0 mg, 4.0-5.0 mg, 2.5-3.0 mg, 3.0-3.5 mg, 3.5-4.0 mg, 4.0-4.5 mg or 4.5-5.0 mg fibrinogen in the film.

In certain embodiments, 0.1-1.0 mL of whole plasma yields 1 cm² of dried film. In certain embodiments, 0.1-0.5 mL, 0.5-1.0 mL, 0.1-0.2 mL, 0.2-0.3 mL, 0.3-0.4 mL, 0.4-0.5 mL, 0.5-0.6 mL, 0.7-0.8 mL, 0.8-0.9 mL 0.9-1.0 mL of whole plasma yields 1 cm² of dried film. In certain embodiments, about 0.1 mL, about 0.2 mL, about 0.3 mL, about 0.4 mL, about 0.5 mL, about 0.6 mL, about 0.7 mL, 0.8 mL, 0.9 mL or about 1.0 mL yields 1 cm² of dried film.

In certain embodiments, the film is less than 1 mm thick. In certain embodiments, the film is 1 mm-5 mm, 0.1-1.5 mm, 0.1-1.0 mm, 0.5-1.0 mm, or 0.1-0.5 mm thick. In certain embodiments, the film is 0.1-0.3 mm thick. In certain embodiments, the film is 0.1-0.2 mm thick. In certain embodiments, the film is 0.15-0.2 mm thick. In certain embodiments, the film has a thickness of 0.1-0.3 mm, 0.1-0.2 mm, or 0.15-0.2 mm.

In certain embodiments, the films have both mechanical strength and elasticity. In certain embodiments, the films have an elastic modulus allowing for 1.5 times elasticity, and a burst pressure of greater than 800 mmHg.

In certain embodiments, the film will degrade post-implantation in less than 50 days. In certain embodiments, the film will degrade post-implantation in less than 45 days, 40 days, 35 days, 30 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days or 2 days. In certain embodiments, the film will degrade post-implantation in less than 28 days. In certain embodiments, the film will degrade post-implantation in less than 21 days.

In certain embodiments, the films are incorporated with various compounds with controlled release properties, for example the described films are loaded with silver microparticles which may be continuously released post implantation until the films degrade.

In certain embodiments, the films further comprise silver. In certain embodiments, the silver can be in the form of, metallic silver microparticles (mean diameter 2 μm to 1,000 μm), silver compounds (silver carbonate, silver chloride, silver phosphate, silver fluoride, silver acetate, silver sulfate, silver citrate, silver oxide) or nanosilver (mean diameter≤2 μm), or combinations thereof.

Silver microparticles of different sizes are generally available from multiple commercial vendors, such as 5 μm (Silpowder 225, Technic, Inc), 15 μm (Atomized fine silver powder 81-800, Technic, Inc.), and 45 μm (Silver powder 327093, Sigma Chemicals).

In certain embodiments, the concentration of silver in the film is a total concentration of Ag>2 nM (2.0×10⁻⁹ M). In certain embodiments the concentration of metallic silver is 2 nM-1 μM, 2 nM-100 nM, 2 nM-10 nM, 10 nM-100 nM, 100 nM-1 μM, 5 nM-10 nM, 10 nM-20 nM, 20 nM-50 nM, 2 nM-5 nM or 500 nM-1 μM. In certain embodiments, the soluble concentration of silver in the film is about 2 nM, about 5 nM, about 10 nM, about 15 nM, about 20 nM, about 30 nM, about 40 nM, about 50 nM, about 50 nM, about 70 nM, about 80 nM, about 90 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM about 950 nM, or about 1 μM.

In certain embodiments, film has a weight/weight ratio of silver to fibrin/fibrinogen of 0.01 (wt/wt) or more, 0.1 (wt/wt) or more, or 0.2 (wt/wt) or more, or 0.3 (wt/wt) or more, or 0.4 (wt/wt) or more, or 0.5 (wt/wt) or more, or 0.6 (wt/wt) or more, or 0.7 (wt/wt) or more, or 0.8 (wt/wt) or more, or 0.9 (wt/wt) or more, or 1 (wt/wt) or more, or 1.1 (wt/wt) or more, or 1.2 (wt/wt) or more, or 1.3 (wt/wt) or more, or 1.4 (wt/wt) or more, or 1.5 (wt/wt) or more, or 1.6 (wt/wt) or more, or 1.7 (wt/wt) or more, or 1.8 (wt/wt) or more, or 1.9 (wt/wt) or more, or 2 (wt/wt) or more, or 2.1 (wt/wt) or more, or 2.2 (wt/wt) or more, or 2.3 (wt/wt) or more, or 2.4 (wt/wt) or more, or 2.5 (wt/wt) or more, or 2.6 (wt/wt) or more, or 2.7 (wt/wt) or more, or 2.8 (wt/wt) or more, or 2.9 (wt/wt) or more, or 3 (wt/wt) or more, or 4 (wt/wt) or more, or 5 (wt/wt) or more, or 10 (wt/wt) or more. For example, an effective amount of the pharmaceutical composition may include a weight/weight ratio of silver to fibrin/fibrinogen of 2.2 (wt/wt) or more. In certain embodiments, an effective amount of the pharmaceutical composition may include a weight/weight ratio of silver to fibrin/fibrinogen of about 1.5 (wt/wt), about 1.6 (wt/wt), about 1.7 (wt/wt), about 1.8 (wt/wt), about 1.9 (wt/wt), 2.0 (wt/wt), about 2.1 (wt/wt), about 2.2 (wt/wt), about 2.3 (wt/wt), about 2.4 (wt/wt), about 2.5 (wt/wt), about 2.6 (wt/wt), about 2.7 (wt/wt), about 2.8 (wt/wt), about 2.9 (wt/wt), about 3.0 (wt/wt), about 3.1 (wt/wt), about 3.2 (wt/wt), about 3.3 (wt/wt), about 3.4 (wt/wt), about 3.5 (wt/wt), about 3.6 (wt/wt), about 3.7 (wt/wt), about 3.8 (wt/wt), about 3.9 (wt/wt), about 4.0 (wt/wt), or about 5.0 (wt/wt).

In certain embodiments, the film releases between 0.0002 and 50.0 ppm silver ions when applied to a subject. In certain embodiments, the film releases between 0.02 and 5.0 ppm silver ions when applied to a subject. In certain embodiments, the film releases between 0.0001-0.001, 0.001-0.01, 0.01-0.1, 0.1-1.0, 0.001-0.005, 0.005-0.01, 0.01-0.05, 0.05-0.10, 0.10-0.15, 0.15-0.20, 0.20-0.25, 0.25-0.30, 0.30-0.35, 0.35-0.40, 0.40-0.45, 0.45-0.50, 0.50-0.55, 0.55-0.60, 0.60-0.65, 0.65-0.70, 0.70-0.75, 0.75-0.80, 0.80-0.85, 0.85-0.90, 0.90-0.95, 0.95-0.10, 1-2, 2-3, 3-4, 4-5, 5-10, 10-20, 20-30, 30-40, or 40-50 ppm silver ions when applied to a subject.

In certain embodiments, the silver is in the form of particles that are substantially solid. Substantially solid particles may, in some instances, be porous (e.g., micro-porous, nano-porous, etc.). However, substantially solid particles do not encompass hollow particles that have a void space surrounded by shell. In these embodiments, the silver particles do not include hollow particles. In certain instances, the silver particles do not include a polymeric material. For example, the silver particles may include only silver (e.g., silver, silver oxide, silver ions, etc.).

In certain embodiments, the silver is in the form of microparticles or nanoparticles. In certain embodiments, the microparticles or nanoparticles are metallic silver microparticles or metallic silver nanoparticles. In certain embodiments the microparticles or nanoparticles comprising silver are not metallic silver but silver in another form (e.g., silver carbonate, silver chloride, silver phosphate, silver fluoride, silver acetate, silver sulfate, silver citrate, silver oxide, or combinations thereof).

In certain embodiments, the silver microparticles have a mean diameter range of 2 μm to 1,000 μm. In certain embodiments, the silver microparticles have a mean diameter range of 10 μm to 1,000 μm, 100 μm to 1,000 μm, 1 μm to 1,000 μm, 10 μm to 100 μm, 500 μm to 1,000 μm, 1 μm to 10 μm, or 10 to 500 μm. In some embodiments, the silver microparticles have a mean diameter range of 10-20 μm. In some embodiments, the silver microparticles have a mean diameter of about 2 μm, 7 μm, 10 μm, 15 μm, 20 μm, or 25 μm. In some embodiments, the silver microparticles have a mean diameter of about 15 μm.

In certain embodiments, the amount of metallic silver in the silver microparticles is present in the film is 0.1-50 mg silver/cm² of film. In certain embodiments, the amount of metallic silver present in the film is 10-50 mg silver/cm², 1-5 mg silver/cm², 5-10 mg silver/cm², 10-15 mg silver/cm², 15-20 mg silver/cm², 20-25 mg silver/cm², 25-30 mg silver/cm², 30-35 mg silver/cm², 35-40 mg silver/cm², 45-50 mg silver/cm², 10-20 mg silver/cm², 20-30 mg silver/cm², 30-40 mg silver/cm², or 40-50 mg silver/cm² In certain embodiments the silver microparticles present at a concentration of about 5 mg silver/cm², about 10 mg silver/cm², about 15 mg silver/cm², about 20 mg silver/cm², about 25 mg silver/cm², about 30 mg silver/cm², about 35 mg silver/cm², about 40 mg silver/cm², about 45 mg silver/cm², or about 50 mg silver/cm².

In certain embodiments, the film comprises about 0.1-50 mg silver microparticles/cm² of film. In certain embodiments, the film comprises about 10-50 mg silver microparticles/cm², 1-5 mg silver microparticles/cm², 5-10 mg silver microparticles/cm², 10-15 mg silver microparticles/cm², 15-20 mg silver microparticles/cm², 20-25 mg silver microparticles/cm², 25-30 mg silver microparticles/cm², 30-35 mg silver microparticles/cm², 35-40 mg silver microparticles/cm², 45-50 mg silver microparticles/cm², 10-20 mg silver microparticles/cm², 20-30 mg silver microparticles/cm², 30-40 mg silver microparticles/cm², or 40-50 mg silver microparticles/cm². In certain embodiments the film comprises about 5 mg silver microparticles/cm², about 10 mg silver/cm², about 15 mg silver microparticles/cm², about 20 mg silver microparticles/cm², about 25 mg silver microparticles/cm², about 30 mg silver microparticles/cm², about 35 mg silver microparticles/cm², about 40 mg silver microparticles/cm², about 45 mg silver microparticles/cm², or about 50 mg silver microparticles/cm².

In some instances, the size of the silver microparticles is sufficient to induce a foreign body reaction in the subject at the site of application. For example, microparticles have an average diameter ranging from 2 μm to 1000 μm. In comparison, nanoparticles have an average diameter of 1 nm to 1000 nm. In some instances, the silver microparticles have an average diameter of 2 μm or more, such as 3 μm or more, including 4 μm or more, or 5 μm or more, or 7 μm or more, or 10 μm or more, or 15 μm or more, or 20 μm or more, or 25 μm or more, or 50 μm or more, or 75 μm or more, or 100 μm or more, or 150 μm or more or 200 μm or more, or 250 μm or more or 500 μm or more. For example, the silver microparticles may have an average diameter ranging from 2 μm to 1000 μm, such as from 2 μm to 750 μm, including from 3 μm to 500 μm, or from 5 μm to 250 μm. In certain instances, the silver microparticles have an average diameter of 5 μm or more. In some cases, the silver microparticles have an average diameter of 200 μm or more. In some embodiments, the silver microparticles include a mixture of silver microparticles having a range of different sizes in the sizes as described above. As used herein, the term “average” is the arithmetic mean.

In certain instances, the silver particles have a substantially symmetrical shape. For example, the silver particles may have a shape that is substantially spherical, elliptical, cylindrical, and the like. In some embodiments, the silver particles have a substantially spherical shape. In other embodiments, the silver particles may have an irregular shape. In certain cases, the silver particles have a substantially smooth outer surface. In other cases, the silver particles have a textured (e.g., rough) outer surface. In some cases, the silver particles include a mixture of silver particles having different shapes and/or textures as described above. In certain instances, the silver particles have a shape and/or texture sufficient to induce a foreign body reaction in the subject at the site of application. For instance, the silver particles may have a spherical shape, a rod shape, a star shape, an irregular shape, combinations thereof, and the like.

In certain embodiments, the film further comprises thrombin. In certain embodiments, the film comprises at least 2 IU/ml thrombin per cm² of film. In certain embodiments, film has more than 2.5 IU thrombin per cm² of film. In certain embodiments, the film 2-2,500 IU, 2-10 IU, 2-100 IU, 100-200 IU, 200-500 IU, 500-1,000 IU, or 1,000-2,500 IU thrombin per cm² of film. In certain embodiments, the film comprises less than 0.1 IU thrombin per cm² of film. In certain embodiments, the film comprises less than 0.105 IU, 0.1 IU, 0.095 IU, 0.09 IU, 0.05 IU, 0.01 IU, 0.009 IU, 0.005 IU, or 0.001 IU thrombin per cm² of film. In certain embodiments, the film does not comprise thrombin.

In certain embodiments, the film comprises whole plasma, and the plasma within the film further comprises thrombin. In certain embodiments, the whole plasma has at least 2 IU/ml thrombin per ml of plasma. In certain embodiments, the whole plasma has more than 2.5 IU thrombin per ml of plasma. In certain embodiments the film comprises whole plasma with 2-2,500, 2-10 IU, 2-100 IU, 100-200 IU, 200-500 IU, 500-1,000 IU, or 1,000-2,500 IU IU thrombin per ml of plasma. In certain embodiments, the whole plasma has less than 0.105 IU/ml, 0.1 IU/ml, 0.095 IU/ml, 0.09 IU/ml, 0.05 IU/ml, 0.01 IU/ml, 0.009 IU/ml, 0.005 IU/ml, or 0.001 IU/ml thrombin per ml of plasma.

In certain embodiments, the film further comprises calcium. In certain embodiments, the film comprises 0.01-1 mg CaCl₂/cm² of film. In certain embodiments, the film comprises 0.01-0.10, 0.01-0.02, 0.02-0.03, 0.03-0.04, 0.04-0.05, 0.05-0.06, 0.06-0.07, 0.07-0.08, 0.09-0.10, 0.1-0.2, 0.2-0.3, 0.3-0.4, 0.4-0.5, 05-0.6, 0.6-0.7, 0.7-0.8, 0.8-0.9, 0.9-1.0 mg/CaCl₂/cm² of film.

In certain aspects, the dried films described herein are available upon release from the storage container comprising the dried film. In certain embodiments, the dried films described herein are available for use and do not require any further components or additives prior to use. In certain embodiments, the dried films described herein can be used within 5 minutes, 4 minutes, 3 minutes, 2 minutes, 1 minute, 45 seconds, 30 seconds, 20 seconds, 10 seconds 5 seconds, or immediately upon release from the storage container comprising the dried film.

In certain aspects, the dried films described herein are stable for up to 3 years. For example, the dried film can be stable for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months, or more. In one aspect, stability is maintenance of at least 80% of a quantitative metric, such as burst pressure, fold number, or fold endurance after a selected amount of time as compared to a newly synthesized film. In some aspects, stability can be the quantitative metric alone or in combination with another quantitative metric. In one embodiment, stability is the combination of burst pressure and fold endurance. Exemplary stability metrics are a burst pressure of at least 800 mm Hg and a fold endurance of at least 100. In some embodiments, stability metrics are a burst pressure of at least 800 mm Hg and a fold endurance of at least 100. In some embodiments, stability metrics are a burst pressure of at least 800 mm Hg and a fold endurance of at least 500. In some embodiments, stability metrics are a burst pressure of at least 800 mm Hg and a fold endurance of at least 1000.

Silver microparticle-containing film stability can be monitored according to the International Council for Harmonization of Technical Requirements for Pharmaceuticals for Human Use (ICH) Q1A (R2): Stability Testing of New Drug Substances and Products, at two conditions: 1. Condition A (2-8° C.) evaluates stability at the temperature at which the drug product (silver microparticle-containing film) is stored; and 2. Condition B (23-27° C./55-65% relative humidity [RH]) assesses effects, if any, of a controlled room temperature hold on the drug product (silver microparticle-containing film). The silver microparticle-containing film can be stored at 2-8° C. and at 23-27° C./55-65% relative humidity for up to 3 years. Stability of the film can be assessed by determining burst pressure, fold number, and fold endurance according to industry standard procedures at least 1 month, 3 months, 6 months, 9 months, 12 months, 18 months, 24 months, 30 months, and 36 months. Burst pressure can be determined by an in-house method according to the ASTM Standard Test Method for Burst Pressure. Strength of Surgical Sealants (F 2392-04). In some embodiments, the flexible plasma-based films have a burst pressure of about 50 to 1000 mm Hg.

Fold number to determine film flexibility is the evaluation of a films fold number. Such determination is accomplished by folding one half of the film on top of the other half, turning the stack by 90° and again folding one half of the stack on top of the other and so on until the film breaks or rupture. Each folding without triggering film rupture increases the fold number by 1. This test is particularly useful for the assessment of a plasma-based film's ability to maintain its integrity when being bent in tight turns. In some embodiments, the flexible plasma-based films of the present invention are have a fold number of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50.

Another test to determine film flexibility is the evaluation of fold endurance. Such determination is accomplished by repeatedly folding one half of the film on top of the other half and unfolding to its original position. Fold endurance is expressed by the number of such folding/unfolding repeats and provides a means for wear resistance estimation. In some embodiments, the flexible plasma-based films of the present invention are have a fold endurance of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 or more or more.

In another aspect, the silver or silver microparticles on the surface of the film are abrasion resistant. As used herein, “abrasion resistant” or “abrasion resistance” is the lack of a material amount of silver or silver microparticle residue that flakes or is brushed off the film after drying. Abrasion resistance can be determined by weighing a silver-containing film, brushing the film with a brush or tool repeatedly, and weighing the film again post-brushing. The amount of silver removed by the abrasion can be assessed as the total weight or percentage of weight lost. In some embodiments, the material amount is less than 0.1%, less than 0.5%, less than 1%, less than 2%, less than 3%, less than 4%, or less than 5% of the total amount of silver applied to the film during production. In some embodiments, the material amount is less than 0.25 mg/cm², less than 0.5 mg/cm², less than 0.75 mg/cm², less than 1 mg/cm², or less than 1.25 mg/cm² of the total mg amount of silver applied to the film during production.

In certain aspects, the dried films described herein have a moisture content (water content) of between about 0.5 to 2.0 mg/cm². For example, the dried film can have a moisture content of about 0.5-2.0 mg/cm², 0.5-1.0 mg/cm², 0.5-0.9 mg/cm², 0.7-0.9 mg/cm², 0.7-1.0 mg/cm², 0.7-1.1 mg/cm², 0.7-1.2 mg/cm², 0.7-1.5 mg/cm², 0.7-1.8 mg/cm², 0.7-2.0 mg/cm², 0.8-0.9 mg/cm², 0.8-1.0 mg/cm², 0.8-1.1 mg/cm², 0.8-1.2 mg/cm², 0.8-1.5 mg/cm², 0.8-1.8 mg/cm², 0.8-2.0 mg/cm², 0.9-1.0 mg/cm², 0.9-1.1 mg/cm², 0.9-1.2 mg/cm², 0.9-1.5 mg/cm², 0.9-1.8 mg/cm², 0.9-2.0 mg/cm², 1.0-1.1 mg/cm², 1.0-1.2 mg/cm², 1.0-1.5 mg/cm², 1-1.8 mg/cm², 1.0-2.0 mg/cm², 1.2-1.5 mg/cm², 1.2-1.8 mg/cm², 1.2-2.0 mg/cm², 1.5-1.8 mg/cm², 1.5-2 mg/cm², or 1.8-2 mg/cm². In some embodiments, the dried film has a moisture content of about 0.5 mg/cm², 0.6 mg/cm², 0.7 mg/cm², 0.8 mg/cm², 0.9 mg/cm², 1.0 mg/cm², 1.1 mg/cm², 1.2 mg/cm², 1.3 mg/cm², 1.4 mg/cm², 1.5 mg/cm², 1.6 mg/cm², 1.7 mg/cm², 1.8 mg/cm², 1.9 mg/cm², or 2.0 mg/cm². In some embodiments, the dried film has a moisture content of about 0.9 mg/cm². Moisture content of the film can be assessed by weighing the dried film, drying the film in an oven for an additional amount of time such as 3 hours at 80° C., and then weighing the film again to determine weight loss due to moisture evaporation.

In certain aspects, the dried films described herein have a moisture content (water content) of between about 2% to 5%. For example, the dried film can have a moisture content of between about 2%-2.5%, 2%-3%, 2%-3.5%, 2%-4%, 2%-4.5%, 2%-5%, 3%-3.3%, 3%-3.5%, 3%-4%, 3%-4.5%, 3%-5%, 4%-4.5%, or 4%-5%. In some embodiments, the dried film has a moisture content of about 2%, 2.3%, 2.5%, 2.7%, 3%, 3.3%, 3.5%, 3.7%, 4%, 4.3%, 4.5%, 4.7%, or 5%. In some embodiments, the dried film has a moisture content of about 3.3%.

In some embodiments, the dried film containing silver microparticles also comprises at least one phospholipid. The phospholipid can be added during the polymerization step. Exemplary phospholipids are phosphatidylserine, phosphatidylcholine, and phosphatidylethanolamine. In some embodiments, the film comprises phosphatidylserine. In some embodiments, the film comprises phosphatidylcholine. In some embodiments, the film comprises phosphatidylethanolamine. In some embodiments, the film comprises phosphatidylserine and phosphatidylcholine.

The silver, such as silver microparticles, can be distributed evenly throughout the film or unevenly throughout the film. In some embodiments, the silver, such as silver microparticles, is distributed evenly throughout the thickness of the film. In some embodiments, the silver, such as silver microparticles, is distributed unevenly throughout the thickness of the film. For instance, there can be a higher amount of silver closer to the top and/or bottom surface of the film and a lower amount of silver closer to the center of the film, forming a concentration gradient. Exemplary concentration gradients (as a percentage of total silver in the film) include, but are not limited to, 99%-1% (surface to center), 97%-3% (surface to center), 95%-5% (surface to center), 90%-10% (surface to center), 85%-15% (surface to center), 80%-20% (surface to center), 75%-25% (surface to center), 70%-30% (surface to center), 65%-35% (surface to center), 60%-40% (surface to center), and 55%-45% (surface to center). The film can have a higher amount of silver microparticles in the surface top and/or bottom 5%, 10%, 15%, 20%, 30% or 40% thickness of the film. For example, in a 1 mm film, if the film has a higher amount of silver, such as silver microparticles, in the top and bottom 0.1 mm, then the film has a higher amount of silver in the surface top and bottom 10% of the film's thickness.

Films Comprising Additional Agents

A dried film described herein can be administered alone or in combination with other treatments, either simultaneously or sequentially dependent upon the condition to be treated.

In certain embodiments, the film includes additional components, such as, but not limited to, a fibrinolysis inhibitor, albumin (e.g., human albumin), tri-sodium citrate, histidine, niacinamide, polysorbate 80, water (e.g., sterile water, such as water for injection), calcium chloride, sodium chloride, combinations thereof, and the like. For example, the first material precursor agent may include, in addition to fibrinogen, one or more of a fibrinolysis inhibitor, albumin (e.g., human albumin), tri-sodium citrate, histidine, niacinamide, polysorbate 80, water (e.g., sterile water, such as water for injection), and the like. In some instances, the second material precursor agent may include, in addition to thrombin, one or more of albumin (e.g., human albumin), water (e.g., sterile water, such as water for injection), calcium chloride, sodium chloride, and the like. Additional examples of components that may be included in the film include but are not limited to, protease inhibitors, such as aprotinin. In certain embodiments, where the film is derived from whole blood or plasma, the film comprises additional components that are present in the blood or plasma at the time of obtaining the blood or plasma to generate the film.

Films Comprising An Adhesive Coating

In certain embodiments, the dried films described herein further comprise an adhesive coating. The adhesive coating can be made of any suitable material that increases adhesion, including, but not limited to cellulose. CMC of low, medium or high viscosity from Sigma can also be used in place of regenerated cellulose. In certain embodiments, the adhesive coating comprises cellulose. In certain embodiments, the adhesive coating comprises oxidized regenerated cellulose. In certain embodiments, the adhesive coating comprises carboxymethyl cellulose. In certain embodiments, the adhesive coating is present in the film at an amount of 1%-25% by weight, 10%-20% by weight, 5%-10% by weight, 10%-15% by weight, 15%-20% by weight, 20%-25% by weight, 5%-20% by weight, or 15%-25% by weight. In certain embodiments, the adhesive coating is present in the film at an amount of about 5% by weight, 6% by weight, 7% by weight, 8% by weight, 9% by weight, 10% by weight, 11% by weight, 12% by weight, 13% by weight, 14% by weight, 15% by weight, 16% by weight, 17% by weight, 18% by weight, 19% by weight, 20% by weight, 21% by weight, 22% by weight, 23% by weight, 24% by weight, or 25% by weight.

Films Comprising Cerium

In certain aspects, provided herein are dried films comprising cerium. In certain embodiments, the dried films comprise silver microparticles and cerium. In certain embodiments, the cerium is present in the polymerized gel prior to removal of the fluid to generate the film at a concentration of 10-500 mM, 20-400 mM, 10-100 mM, 20-50 mM, 50-100 mM, 100-150 mM, 150-200 mM, 200-250 mM, 250-300 mM, 300-350 mM, 350-400 mM, or 400-500 mM. In certain embodiments, the cerium is present in the polymerized gel prior to removal of the fluid to generate the film at a concentration of about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 320 mM, about 330 mM, about 340 mM, about 350 mM, about 360 mM, about 370 mM about 380 mM, about 390 mM or about 400 mM.

In certain embodiments, the dried films comprising cerium further comprise silver. In certain embodiments, the silver is in the form of silver nanoparticles or silver microparticles. In certain embodiments, the silver is in the form of metallic silver nanoparticles or metallic silver microparticles. In certain embodiments, the dried films comprising cerium and silver microparticles comprise 10-50 mg silver microparticles/cm² film. In certain embodiments, the dried films comprising cerium further comprise silver microparticles present in the film at 10-50 mg silver/cm², 1-5 mg silver/cm², 5-10 mg silver/cm², 10-15 mg silver/cm², 15-20 mg silver/cm², 20-25 mg silver/cm², 25-30 mg silver/cm², 30-35 mg silver/cm², 35-40 mg silver/cm², 45-50 mg silver/cm², 10-20 mg silver/cm², 20-30 mg silver/cm², 30-40 mg silver/cm², or 40-50 mg silver/cm² wound surface area. In certain embodiments the silver microparticles are present at a concentration of about 5 mg silver/cm², about 10 mg silver/cm², about 15 mg silver/cm², about 20 mg silver/cm², about 25 mg silver/cm², about 30 mg silver/cm², about 35 mg silver/cm², about 40 mg silver/cm², about 45 mg silver/cm², or about 50 mg silver/cm² of film.

Films From Whole Blood

In certain aspects, the dried films described herein comprise whole blood. In certain aspects the dried films comprising whole blood is autologous whole blood, intended to be used for treating or preventing a wound in a patient.

As an example, a film can be made immediately after patient blood collection via the methods described herein. An exemplary method is: mix the patient blood with calcium and/or thrombin to form a clot (gel), place the clot (gel) on a small vacuum fixture and apply low vacuum for 5 minutes. Silver can be added before or after the vacuum step, and the film can then be placed on the wound. Such production can be done patient side, resulting in a process that can be started and completed with placement of the film on the wound in less than 10 minutes. Small films can be made with as little as 10 mL of whole blood from the donor or patient.

In certain embodiments the dried films described herein comprise whole blood and further comprise silver. In certain embodiments the dried films described herein comprise whole blood and further comprise silver in the form of microparticles or nanoparticles. In certain embodiments the dried films described herein comprise whole blood and further comprise silver in the form of metallic silver microparticles or metallic silver nanoparticles. In certain embodiments, the dried films from whole blood further comprise silver microparticles present in the film at 10-50 mg silver/cm², 1-5 mg silver/cm², 5-10 mg silver/cm², 10-15 mg silver/cm², 15-20 mg silver/cm², 20-25 mg silver/cm², 25-30 mg silver/cm², 30-35 mg silver/cm², 35-40 mg silver/cm², 45-50 mg silver/cm², 10-20 mg silver/cm², 20-30 mg silver/cm², 30-40 mg silver/cm², or 40-50 mg silver/cm² wound surface area. In certain embodiments the silver microparticles are present at a concentration of about 5 mg silver/cm², about 10 mg silver/cm², about 15 mg silver/cm², about 20 mg silver/cm², about 25 mg silver/cm², about 30 mg silver/cm², about 35 mg silver/cm², about 40 mg silver/cm², about 45 mg silver/cm², or about 50 mg silver/cm² of film.

Methods for Enhancing Wound Healing and Treating Wounds

In certain aspects, the methods described herein are for treating wound and enhancing the healing of various wounds, including, but not limited to, acute wounds, chronic wounds, non-healing wounds, diabetic foot ulcers, venous leg ulcers, pressure ulcers, arterial wounds, ulcers, abdominal incisional wounds, surgical wounds, traumatic wounds, lipocutaneous flaps and skin disorders defined by ICD-9 code. International Classification of Diseases, Ninth Revision (ICD-9) codes are available from the CDC at cdc.gov/nchs/icd/icd9

The frequency with which the invention is applied depends on the clinical setting of the wound under treatment, and can range from a single application when treating abdominal incisional wounds or lipocutaneous flaps to multiple applications coincident with wound dressing changes when treating ulcers, surgical wounds, traumatic wounds, burns or skin disorders. In certain aspects, the methods described herein enhance the healing of injured tissues and cells, including decreased primary post-surgical adhesion formation, post-surgical adhesion reformation, and cellular and tissue engraftment (e.g., pancreatic islet or hepatocellular (liver cell) transplant survival).

In some instances, enhancing healing of a wound includes an increase in the efficiency of wound healing and/or an increase in the strength of the resulting healed wound site as compared to a wound that has not been treated with the methods and compositions of the present disclosure. In some cases, enhancing healing of a wound includes reducing the occurrence of defective wound healing and/or reducing the severity of defective wound healing as compared to a wound that has not been treated with the methods and compositions of the present disclosure.

In certain aspects, the methods and silver-containing films described herein induce formation of foreign body giant cells. Without wishing to be bound by theory, it is proposed that in some embodiments wherein the film comprises silver microparticles larger than 5 the silver microparticles cannot be phagocytosed by cells such as macrophages. This is proposed to result in fusion of macrophage cells and the formation of multinucleated foreign body giant cells. Without wishing to be bound by theory, such cells then secrete various cell factors and cytokines, such as IL-1α, IL-1β, IL-10TNF-α, TGF-β, IFN-γ, matrix metalloproteases (MMPs,) MMP-9, TIMP-1, TIMP-2, as well as secrete growth and angiogenic factors. Without wishing to be bound by theory, foreign body giant cells also stimulate fibroblast activity, overexpress extracellular matrix proteins, and recruit cells that secrete collagen. Thus, without wishing to be bound by theory, the inclusion of silver microparticles that are too large to be phagocytosed by macrophages results in increased wound healing. In some embodiments, the silver microparticles that are too large to be phagocytosed by macrophages at least 5 μm, 7 μm, 10 μm, 12 μm, 15 μm, 17 μm, 20 μm, 25 μm, or 30 or larger in size. In some embodiments, the silver microparticles that are too large to be phagocytosed by macrophages at least 15 μm in size.

In some instances, the methods described herein reduce the risk of incisional hernia by 30% or more, such as 35% or more, including 40% or more, or 45% or more, or 50% or more, or 55% or more, or 60% or more, or 65% or more, or 70% or more, or 75% or more, or 80% or more, or 85% or more, or 90% or more, or 95% or more, for example by 99% or more. In certain cases, the method reduces the risk of incisional hernia by 60% or more. For example, the method may reduce the risk of a clinical hernia in a subject. By “reduce the risk,” it is meant that the risk of the occurrence of incisional hernia in a subject treated by the method of the present disclosure is lower than that in a subject that has not been treated by the method of the present disclosure. By “clinical hernia,” it is meant a hernia that is observed (e.g., by sight, touch, sound, smell, etc.) during treatment of a patient, rather than determined through laboratory studies. For example, a clinical hernia may be observed as a visible bulge in the abdominal wall.

In certain embodiments, the method for treating a wound in a subject reduces severity of a hernia in a subject should a hernia occur in the subject. In some cases, a reduction in the severity of the hernia corresponds to a reduction in the size of the hernia in the subject. For example, the method may reduce the size of an anatomic hernia in a subject. By “anatomic hernia,” it is meant a hernia that is detectable by methods other than, or in addition to, clinical observation (e.g., by dissection of the subject, MRI, CT, ultrasound, and the like). The size of an anatomic hernia may be measured by determining the separation between the abdominal muscles (e.g., rectus muscles) at the incision site. For instance, the size of an anatomic hernia may be estimated by multiplying the maximal craniocaudal diameter by the average of two transverse diameter measurements (e.g., approximation of an ellipse). In some instances, the method reduces the size of incisional hernia by 15% or more, such as 20% or more, including 25% or more, or 30% or more, such as 35% or more, including 40% or more, or 45% or more, or 50% or more, or 55% or more, or 60% or more, or 65% or more, or 70% or more, or 75% or 45 more, or 80% or more, or 85% or more, or 90% or more, or 95% or more, for example by 99% or more. In certain cases, the method reduces the risk of incisional hernia by 55% or more.

The fibrinogen/fibrin films described herein that are to be given to an individual, administration is preferably in a “therapeutically effective amount” of a pharmaceutical composition that is sufficient to show benefit to the individual. By “effective amount” is meant a dosage sufficient to cause a significantly detectable effect in the target subject, as desired. In some instances, an effective amount of the pharmaceutical composition is an amount of the pharmaceutical composition sufficient to induce a foreign body reaction in the subject at the site of application. In certain instances, the foreign body reaction includes inflammatory infiltrate consisting of giant cells without epitheliod histiocytes.

In certain embodiments, an effective amount of the pharmaceutical composition includes a weight/weight ratio of silver to fibrinogen of 0.1 (wt/wt) or more, or 0.2 (wt/wt) or more, or 0.3 (wt/wt) or more, or 0.4 (wt/wt) or more, or 0.5 (wt/wt) or more, or 0.6 (wt/wt) or more, or 0.7 (wt/wt) or more, or 0.8 (wt/wt) or more, or 0.9 (wt/wt) or more, or 1 (wt/wt) or more, or 1.1 (wt/wt) or more, or 1.2 (wt/wt) or more, or 1.3 (wt/wt) or more, or 1.4 (wt/wt) or more, or 1.5 (wt/wt) or more, or 1.6 (wt/wt) or more, or 1.7 (wt/wt) or more, or 1.8 (wt/wt) or more, or 1.9 (wt/wt) or more, or 2 (wt/wt) or more, or 2.1 (wt/wt) or more, or 2.2 (wt/wt) or more, or 2.3 (wt/wt) or more, or 2.4 (wt/wt) or more, or 2.5 (wt/wt) or more, or 2.6 (wt/wt) or more, or 2.7 (wt/wt) or more, or 2.8 (wt/wt) or more, or 2.9 (wt/wt) or more, or 3 (wt/wt) or more. For example, an effective amount of the pharmaceutical composition may include a weight/weight ratio of silver to fibrinogen of 2.2 (wt/wt) or more.

In certain embodiments, an effective amount of the pharmaceutical composition includes an amount of silver, as described herein, applied to a certain wound surface area, such as 0.5 cm² or more, or 1 cm² or more, or 2 cm² or more, or 3 cm² or more, or 4 cm² or more, or 5 cm² or more, or 6 cm² or more, or 7 cm² or more, or 8 cm² or more, or 9 cm² or more, or 10 cm² or more, or 11 cm² or more, or 12 cm² or more, or 13 cm² or more, or 14 cm² or more, or 15 cm² or more, or 16 cm² or more, or 17 cm² or more, or 18 cm² or more, or 19 cm² or more, or 20 cm² or more, or 25 cm² or more, or 30 cm² or more, or 35 cm² or more, or 40 cm² or more, or 45 cm² or more, or 50 cm² or more. For example, an effective amount of the pharmaceutical composition may include an amount of silver particles, such as 250 mg/mL, applied to a wound surface area of 10 cm² or more. In some instances, an effective amount of the pharmaceutical composition may include an amount of silver particles, such as 2.2 (wt/wt), applied to a wound surface area of 10 cm² or more.

In certain aspects, an effective amount of the pharmaceutical composition includes silver microparticles. In certain cases, an effective amount of the pharmaceutical composition includes 1 mg silver microparticles/cm² film or more, 10 mg silver microparticles/cm² film or more, such as 25 mg silver microparticles/cm² film or more, including 50 mg/silver microparticles/cm² film or more, or 75 mg silver microparticles/cm² film or more, or 100 mg silver microparticles/cm² film or more, or 150 mg silver microparticles/cm² film or more, or 200 mg silver microparticles/cm² film or more, or 250 mg silver microparticles/cm² film or more, or 300 mg silver microparticles/cm² film or more. In certain cases, an effective amount of the pharmaceutical composition includes 10 mg silver microparticles/cm² film or more, such as 25 mg silver microparticles/cm² film or more, including 50 mg silver microparticles/cm² film or more, or 75 mg silver microparticles/cm² film or more, or 100 mg silver microparticles/cm² film or more, or 150 mg silver microparticles/cm² film or more, or 200 mg silver microparticles/cm² film or more, or 250 mg silver microparticles/cm² film or more, or 300 mg silver microparticles/cm² film or more, or 350 mg silver microparticles/cm² film or more, or 400 mg silver microparticles/cm² film or more, or 450 mg silver microparticles/cm² film or more, or 500 mg silver microparticles/cm² film or more, or 550 mg silver microparticles/cm² film or more, or 600 mg silver microparticles/cm² film or more, or 650 mg silver microparticles/cm² film or more, or 700 mg silver microparticles/cm² film or more, or 750 mg silver microparticles/cm² film or more. In certain instances, an effective amount of the pharmaceutical composition includes 50 mg silver microparticles/cm² film. In some cases, an effective amount of the pharmaceutical composition includes 500 mg silver microparticles/cm² film.

Method for Preventing Wounds

Included herein are methods of preventing wounds. In certain embodiments, the methods herein prevent the occurrence of wounds or prevent the exacerbation of existing wounds. In certain aspects, the dried films described herein are applied to an area in the subject that is susceptible to forming a wound in the subject. For example, the films described herein could be applied for the prevention of a hernia, or any soft tissue reinforcement. In certain embodiments, the films provided herein are applied to prevent wounds, such as surgical sounds, from becoming exacerbated. In certain embodiments the films provided herein are used for soft tissue (dead space) management, for example, during surgical procedures.

Methods of Producing Fibrin/Fibrinogen Films

Included herein are methods of producing films comprising fibrin and/or fibrinogen. In certain embodiments, the film is produced by obtaining whole blood or whole plasma, that is optionally virally-inactivated. In certain embodiments, the whole blood or whole plasma is virally inactivated by a suitable solvent/detergent (S/D) treatment and/or nanofiltration to further inactivate/remove viral particles.

In certain embodiments, the films are produced by placing a solution comprising fibrin and/or fibrinogen (e.g., placing plasma) into a mold. In certain embodiments, the solution comprising fibrin and/or fibrinogen is human plasma. In certain embodiments, the solution comprising fibrin and/or fibrinogen is a solution with purified or recombinant purified fibrin and/or fibrinogen.

In certain embodiments, 0.1-1.5 mL of whole plasma yields 1 cm² of dried film. In certain embodiments, 0.1-0.5 mL, 0.5-1.0 mL, 0.1-0.2 mL, 0.2-0.3 mL, 0.3-0.4 mL, 0.4-0.5 mL, 0.5-0.6 mL, 0.7-0.8 mL, 0.8-0.9 mL 0.9-1.0 mL of whole plasma yields 1 cm² of dried film. In certain embodiments, about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL or about 1.5 mL yields 1 cm² of dried film.

In certain embodiments, the solution comprising fibrin and/or fibrinogen is mixed with thrombin. In some embodiments, polymerization of the plasma can be achieved by recalcification of the plasma. CaCl₂ can be added to the solution comprising fibrin and/or fibrinogen to polymerize (clot) the solution and form a gel. In certain embodiments, the solution comprising fibrin and/or fibrinogen is mixed with CaCl₂. In certain embodiments, about 0.01-1 mg CaCl₂/ml of plasma is added to the solution comprising fibrin and/or fibrinogen to polymerize (clot) the solution and form a gel. In certain embodiments, about between 0.01-0.10, 0.01-0.02, 0.02-0.03, 0.03-0.04, 0.04-0.05, 0.05-0.06, 0.06-0.07, 0.07-0.08, 0.09-0.10, 0.1-0.2, 0.2-0.3, 0.3-0.4, 0.4-0.5, 05-0.6, 0.6-0.7, 0.7-0.8, 0.8-0.9, 0.9-1.0 mg CaCl₂/mL of plasma is added to the solution comprising fibrin and/or fibrinogen to polymerize (clot) the solution and form a gel. In some embodiments, about 0.8-0.9 mg CaCl₂/mL of plasma is added. In some embodiments, about 0.85, 0.86, or 0.87 mg CaCl₂/mL of plasma is added.

In addition, the extrinsic pathway of the coagulation system can be activated to induce polymerization of the plasma. Such extrinsic activators are phospholipids such as phosphatidylserine and phosphatidylcholine. In one embodiment, the solution comprising fibrin and/or fibrinogen is mixed with phosphatidylserine. In one embodiment, the solution comprising fibrin and/or fibrinogen is mixed with phosphatidylcholine. In one embodiment, the solution comprising fibrin and/or fibrinogen is mixed with between 0.1-1 mg/ml, 0.1-0.2 mg/ml, 0.2-0.3 mg/ml, 0.3-0.4 mg/ml, 0.4-0.5 mg/ml, 0.5-0.6 mg/ml, 0.6-0.7 mg/ml, 0.7-0.8 mg/ml, 0.8-0.9 mg/ml, or 0.9-1.0 mg/ml phospholipid.

In certain embodiments, the solution comprising fibrin and/or fibrinogen is virally inactivated prior to pouring the solution into the mold. The solution can be virally inactivated by any known method in the art, e.g., solvent-detergent treated. Any suitable solvent-detergent can be used, including but not limited to, Triton-X-100. virally inactivated plasma suitable for use in making the films of the present disclosure is commercially available, such as Octaplas from Octapaharma USA, (Paramus, N.J.).

The solution comprising fibrin and/or fibrinogen (e.g., plasma) is allowed to polymerize into a gel in the mold. In certain embodiments the polymerization of the gel (e.g, plasma) takes place at 37° C. In certain embodiments the polymerization of the solution comprising fibrin and/or fibrinogen (e.g, plasma) takes place at room temperature. The polymerization can take between 5 min to 60 min. In some embodiments, the solution comprising fibrin and/or fibrinogen (e.g., plasma) is incubated for between 5 min and 60 min, such as 5-10 min, 5-15 min, 5-30 min, 5-45 min, 5-60 min, 10-20 min, 10-30 min, 10-45 min, 10-60 min, 15-30 min, 15-45 min, 15-60 min, 30-45 min, 30-60 min, 5, 8, 10, 15, 20, 25, 30, 35, 40, 45, 50, 55, or 60 min. In some embodiments, the solution comprising fibrin and/or fibrinogen (e.g., plasma) is incubated at 37° C. for 5 to 30 min. In some embodiments, the solution comprising fibrin and/or fibrinogen (e.g., plasma) is incubated at 37° C. for 15 min.

Fluid is removed from the polymerized gel to form a film. In certain embodiments the removal of fluid is performed by application of pressure. In certain embodiments, the removal of fluid is performed by use of a vacuum, or centrifugation of the gel.

In certain embodiments, the polymerized gel is subjected to a mechanical pressure (i.e., “pressed”) of 500-5,000 PSI, or 1,000-3,000 PSI, or 1,000-2,000 PSI or 2,000-3,000 PSI for 5-15 min, 1-30 min, 1-60 min, 1 min-12 hours, or 1 min-24 hours. In certain embodiments, the pressed polymerized is subjected to a sufficient pressure for a sufficient length of time to dry the gel into a dried film.

In certain embodiments, the polymerized gel is subjected to a vacuum of between about 1-150 Torr, 1-1.5 Torr, 1.5-125 Torr, 1.5-10 Torr, 10-20 Torr, 20-30 Torr, 30-40 Torr, 40-50 Torr, 50-60 Torr, 60-70 Torr, 70-80 Torr, 80-90 Torr, 90-100 Torr, 100-110 Torr, 110-120 Torr, 120-130 Torr, 130-140 Torr, or 140-150 Torr. In certain embodiments, the polymerized gel is subjected to a vacuum of about 1, 1.5, 2, 3, 4, 5, 6, 7, 8, 9, 10, 20, 30, 40, 50, 60, 70, 80, 90, 120, 130, 140, or 150 Torr for 5-15 min, 1-30 min, 1-60 min, 1 min-12 hours, or 1 min-24 hours. In certain embodiments, the polymerized gel is subjected to a vacuum pressure of 1.5 torr to 125 torr for 15 min. In certain embodiments, the polymerized gel is subjected to a vacuum pressure of about 1, 1.5, 2, 3, 4, 5, 6, 7, 8, 9, 10, 20, 30, 40, 50, 60, 70, 80, 90, 120, 130, 140, or 150 torr for 15 min.

In certain embodiments, the film is incubated in a glycerin or glycerol solution bath. The film can be incubated in the glycerin or glycerol solution bath prior to addition of silver or after addition of silver, such as silver microparticles. The bath can be a 1-5% glycerol solution. For example, the solution can be a 1%, 1.5%, 2%, 2.5%, 3%, 3.5%, 4%, 4.5%, or 5% glycerol solution. The solution can be a 1-5% glycerin solution. For example, the bath can be a 1%, 1.5%, 2%, 2.5%, 3%, 3.5%, 4%, 4.5%, or 5% glycerin solution. In some embodiments, the solution is a 2% glycerin solution. In some embodiments, the solution is a 5% glycerin solution. The film can be direct incubated in the glycerin or glycerol bath, or the glycerin or glycerol solution can be applied to the film while it is still in the vacuum press module and vacuum applied to draw the glycerin or glycerol solution into the film.

In certain embodiments, silver is added prior to drying the film. In certain embodiments, silver is added to the plasma. In certain embodiments, silver is added to the polymerized gel. In certain embodiments, silver is added prior to pressing the solution. In certain embodiments, silver is added after pressing by application of a silver spray coating. In some embodiments, silver is applied via spray coating. In certain embodiments, silver is added prior to applying vacuum pressure to the solution. In certain embodiments, silver is added after applying vacuum pressure to the solution. In certain embodiments, silver is added prior to removing any effluent fluid. In certain embodiments, silver is added after removing any effluent fluid. In certain embodiments, silver is added prior to the glycerin or glycerol solution incubation or bath. In certain embodiments, silver is added after the glycerin or glycerol solution incubation or bath.

In some embodiments, the silver is silver microparticles. In certain embodiments, the silver microparticles are added prior to drying the film. In certain embodiments, the silver microparticles are added to the plasma. In certain embodiments, the silver microparticles are added to the polymerized gel. In certain embodiments, the silver microparticles are added before polymerization of the gel. In certain embodiments, the silver microparticles are added after polymerization of the gel. In certain embodiments, the silver microparticles are added before removal of the fluid from the gel. In certain embodiments, the silver microparticles are added after removal of the fluid from the gel. In certain embodiments, the silver microparticles are added prior to the glycerin or glycerol solution incubation or bath. In certain embodiments, the silver microparticles are added after the glycerin or glycerol solution incubation or bath.

In some embodiments, the silver, such as silver microparticles, can be applied to one side of the film. In some embodiments, the silver, such as silver microparticles, can be applied to both sides of the film. In some embodiments, the silver, such as silver microparticles, can be interspersed or distributed within the film.

In certain embodiments, the silver microparticles have a mean diameter range of 2 μm to 1,000 μm. In certain embodiments, the silver microparticles have a mean diameter range of 10 μm to 1,000 μm, 100 μm to 1,000 μm, 1 μm to 1,000 μm, 10 μm to 100 μm, 500 μm to 1,000 μm, 1 μm to 10 μm, or 10 to 500 μm. In some embodiments, the silver microparticles have a mean diameter range of 10-20 μm. In some embodiments, the silver microparticles have a mean diameter of about 2 μm, 7 μm, 10 μm, 15 μm, 20 μm, or 25 μm. In some embodiments, the silver microparticles have a mean diameter of about 15 μm.

In certain embodiments, the amount of metallic silver in the silver microparticles added to the plasma, gel, or film is 0.1-50 mg silver/cm² of film. In certain embodiments, the amount of metallic silver present added to the plasma, gel, or film is 10-50 mg silver/cm², 1-5 mg silver/cm², 5-10 mg silver/cm², 10-15 mg silver/cm², 15-20 mg silver/cm², 20-25 mg silver/cm², 25-30 mg silver/cm², 30-35 mg silver/cm², 35-40 mg silver/cm², 45-50 mg silver/cm², 10-20 mg silver/cm², 20-30 mg silver/cm², 30-40 mg silver/cm², or 40-50 mg silver/cm². In certain embodiments, the amount of metallic silver present added to the plasma, gel, or film is 10-50 mg silver/ml, 1-5 mg silver/ml, 5-10 mg silver/ml, 10-15 mg silver/ml, 15-20 mg silver/ml, 20-25 mg silver/ml, 25-30 mg silver/ml, 30-35 mg silver/ml, 35-40 mg silver/ml, 45-50 mg silver/ml, 10-20 mg silver/ml, 20-30 mg silver/ml, 30-40 mg silver/ml, or 40-50 mg silver/ml. In certain embodiments the silver microparticles are present at a concentration of about 5 mg silver/cm², about 10 mg silver/cm², about 15 mg silver/cm², about 20 mg silver/cm², about 25 mg silver/cm², about 30 mg silver/cm², about 35 mg silver/cm², about 40 mg silver/cm², about 45 mg silver/cm², or about 50 mg silver/cm² of the plasma, gel, or film. In certain embodiments the silver microparticles are present at a concentration of about 5 mg silver/ml, about 10 mg silver/ml, about 15 mg silver/ml, about 20 mg silver/ml, about 25 mg silver/ml, about 30 mg silver/ml, about 35 mg silver/ml, about 40 mg silver/ml, about 45 mg silver/ml, or about 50 mg silver/ml of the plasma, gel, or film.

In certain embodiments, about 0.1-50 mg silver microparticles/cm² of film or 0.1-50 mg silver microparticles/ml of plasma or gel is added to the plasma, gel, or film. In certain embodiments, about 10-50 mg silver microparticles/cm² of film or mg silver microparticles/ml of plasma or gel, 1-5 mg silver microparticles/cm² of film or mg silver microparticles/ml of plasma or gel, 5-10 mg silver microparticles/cm² of film or mg silver microparticles/ml of plasma or gel, 10-15 mg silver microparticles/cm² of film or mg silver microparticles/ml of plasma or gel, 15-20 mg silver microparticles/cm² of film or mg silver microparticles/ml of plasma or gel, 20-25 mg silver microparticles/cm² of film or mg silver microparticles/ml of plasma or gel, 25-30 mg silver microparticles/cm² of film or mg silver microparticles/ml of plasma or gel, 30-35 mg silver microparticles/cm² of film or mg silver microparticles/ml of plasma or gel, 35-40 mg silver microparticles/cm² of film or mg silver microparticles/ml of plasma or gel, 45-50 mg silver microparticles/cm² of film or mg silver microparticles/ml of plasma or gel, 10-20 mg silver microparticles/cm², 20-30 mg silver silver microparticles/cm² of film or mg silver microparticles/ml of plasma or gel, 30-40 mg silver microparticles/cm² of film or mg silver microparticles/ml of plasma or gel, or 40-50 mg silver microparticles/cm² of film or mg silver microparticles/ml of plasma or gel is added to the plasma, gel, or film. In certain embodiments, about 5 mg silver microparticles/cm², about 10 mg silver/cm², about 15 mg silver microparticles/cm², about 20 mg silver microparticles/cm², about 25 mg silver microparticles/cm², about 30 mg silver microparticles/cm², about 35 mg silver microparticles/cm², about 40 mg silver microparticles/cm², about 45 mg silver microparticles/cm², or about 50 mg silver microparticles/cm² is added to the plasma, gel, or film. In certain embodiments, about 5 mg silver microparticles/ml, about 10 mg silver/ml, about 15 mg silver microparticles/ml, about 20 mg silver microparticles/ml, about 25 mg silver microparticles/ml, about 30 mg silver microparticles/ml, about 35 mg silver microparticles/ml, about 40 mg silver microparticles/ml, about 45 mg silver microparticles/ml, or about 50 mg silver microparticles/ml is added to the plasma, gel, or film.

In certain embodiments, the film generated from the fluid removal is subjected to gamma irradiation. In certain embodiments, the film is stored at room temperature after removal of the fluid. In certain embodiments, the film is stored at room temperature after gamma irradiation.

In certain embodiments, the films and pharmaceutical compositions comprising the films disclosed herein are packaged in a sterile container. In certain embodiments, the pressed polymerized gel is stored at room temperature after gamma irradiation.

In certain embodiment cerium is added to the polymerized gel prior to removal of the fluid to generate the film. In certain embodiments, the cerium is present in the polymerized gel at a concentration of 10-500 mM, 20-400 mM, 10-100 mM, 20-50 mM, 50-100 mM, 100-150 mM, 150-200 mM, 200-250 mM, 250-300 mM, 300-350 mM, 350-400 mM, or 400-500 mM. In certain embodiments, the cerium is present in the polymerized gel at a concentration of about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 320 mM, about 330 mM, about 340 mM, about 350 mM, about 360 mM, about 370 mM about 380 mM, about 390 mM or about 400 mM.

The film can be dried after application of the silver and/or incubation in a glycerin or glycerol bath. In some embodiments, the film is dried in an oven at between 40-70° C., such as between 40-45° C., 45-50° C., 50-55° C., 55-60° C., 60-65° C., or 65-70° C. In some embodiments, the film is dried in an oven at between 42-50° C. In some embodiments, the film is dried in an oven at about 40, 42° C., 45° C., 47° C., 50° C., 55° C., 60° C., 65° C., or 70° C. In some embodiments, the film is dried in an oven at about 42° C. In some embodiments, the film is dried in an oven at about 50° C. The films can be dried for about 15 min to 3 hours. In some embodiments, the films are dried for about 15 min to 30 hours, 30 min to 2 hours, 45 min to 1.5 hours, or about 1 hour. In some embodiments, the films are dried for about 15 min, 30 min, 45 min, 60 min, 75 min, 90 min, 105 min, 120 min, 135 min, 150 min, 165 min, or 180 min.

In certain embodiments an adhesive coating is added to the film.

Kits

Also provided are kits that find use in practicing the subject methods, as described above. For example, kits for practicing the subject methods may include a sterile container containing a dried film described herein in an amount effective to promote healing of a wound (e.g., an abdominal incision site). In certain embodiments, the kits include a sealed package configured to maintain the sterility of the sterile container. The sealed package may be sealed such that substantially no external contaminants, such as dirt, microbes (e.g., fungi, bacteria, viruses, spore forms, etc.), liquids, gases, and the like, are able to enter the sealed package. For example, the sealed package may be sealed such the package is water-tight and/or air-tight.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed in the packaging of the kit, in a package insert, etc. Another means would be a computer readable medium, e.g., CD, DVD, Blu-ray, computer-readable memory, etc., on which the information has been recorded or stored. Yet another means that may be present is a website address which may be used via the Internet to access the information at a removed site. Any convenient means may be present in the kits.

### Additional Embodiments

Disclosed herein are dried films for treating or preventing a wound. In certain aspects, described herein is a dried film, comprising: fibrin, fibrinogen, or combinations thereof; wherein the film is stable at room temperature for at least three years. In certain embodiments, the fibrin or fibrinogen is present as a component of whole blood or whole plasma. In certain embodiments, the fibrin, fibrinogen or combinations thereof are present at a concentration of 0.5 to 20.0 mg/cm² of film. In certain embodiments, the fibrinogen is present at a concentration of 2.5 to 4.0 mg/cm² of film. In certain embodiments, the dried film further comprises silver or a silver-containing compound. In certain embodiments, the silver or silver-containing compound is silver carbonate, silver chloride, silver phosphate, silver fluoride, silver acetate, silver sulfate, silver citrate, silver oxide, or combinations thereof. In certain embodiments, the silver is present in the film at a total concentration of 0.1-50 mg/cm² of film. In certain embodiments, the silver is in the form of nanoparticles or microparticles, or combinations thereof. In certain embodiments, the nanoparticles or microparticles are metallic silver nanoparticles or metallic silver microparticles. In certain embodiments, the metallic silver is present in the film at a total concentration of 0.1-50 mg/cm² of film. In certain embodiments, the silver microparticles have a mean diameter of less than 2 μm. In certain embodiments, the silver microparticles have a mean diameter ranging from 2 μm to 1,000 μm. In certain embodiments, the silver microparticles are present at a concentration of 10-50 mg silver/cm² of film. In certain embodiments, the silver microparticles are present at a concentration of 10-25 mg silver/cm² of film. In certain embodiments, the film releases between 0.0002 and 10.0 ppm silver ions when applied to a subject. In certain embodiments, the film further comprises cerium. In certain embodiments, the cerium is present in the film at a concentration of 20-400 mM. In certain embodiments, the film further comprises an adhesive coating. In certain embodiments, the adhesive coating is oxidized regenerated cellulose. In certain embodiments, the adhesive coating is present in the film at an amount of 5%-25% by weight. In certain embodiments, the film is sterile. In certain embodiments, the film is sterilized by gamma irradiation. In certain embodiments, the film further comprises calcium. In certain embodiments, the calcium is present in the film at a concentration of 0.00005-0.20 mg/cm² of film. In certain embodiments, the film further comprises thrombin. In certain embodiments, the thrombin is present in the film at a concentration of at least 2 IU thrombin per cm² of film. In certain embodiments, the thrombin concentration is greater than 2.5 IU thrombin per cm² of film. In certain embodiments, the thrombin concentration is less than 2,500 IU thrombin per cm² of film. In certain embodiments, the film has a thickness of less than 1 mm.

In certain aspects, disclosed herein is a method of treating a wound in a subject, comprising application of an effective amount of the dried film of any one of claims 1-29 to the wound. In certain embodiments, the dried film is applied to the wound less than 15 minutes after opening a storage container comprising the dried film. In certain embodiments, the dried film is applied to the wound between 1 and 15 minutes after opening the storage container comprising the dried film. In certain embodiments, the wound is a non-healing wound. In certain embodiments, wound is selected from the group consisting of decreased primary post-surgical adhesion formation, decreased post-surgical adhesion reformation, a wound from cellular and tissue engraftment, a burn wound. In certain embodiments, the dried film degrades in less than 21 days after the application of the film to the wound of the subject.

In certain aspects, described herein is a method of manufacturing a dried film for treating wounds in a subject, comprising obtaining human plasma; placing the plasma into a mold; polymerizing the plasma to form a gel; adding a silver-containing compound to the plasma to generate a silver-containing plasma mixture; removing fluid from the polymerized gel to generate a film; and gamma sterilizing the film. In certain embodiments, the removal of fluid is performed by applying pressure to the gel at a pressure of 100-10,000 PSI to generate a film. In certain embodiments, the pressure applied to the gel is about 1,000 PSI. In certain embodiments, the plasma is further mixed with CaCl₂. In certain embodiments, the plasma is further mixed with thrombin. In certain embodiments, the plasma is mixed with CaCl₂ and thrombin prior to placing the plasma into the mold. In certain embodiments, the silver is added after removal of the fluid from the polymerized gel. In certain embodiments, the silver is added by applying a spray coating on the plasma polymerized gel that comprises a silver-containing compound. In certain embodiments, the silver-containing compound is selected from the group consisting of: silver carbonate, silver chloride, silver phosphate, silver fluoride, silver acetate, silver sulfate, silver citrate, and silver oxide. In certain embodiments, the silver is present in the film at a total concentration of 0.1-50 mg/cm² of film. In certain embodiments, the silver is in the form of silver nanoparticles, silver microparticles, or combinations thereof. In certain embodiments, the nanoparticles or microparticles are metallic silver nanoparticles or metallic silver microparticles. In certain embodiments, the metallic silver is present in the film at a total concentration of 0.1-50 mg/cm² of film. In certain embodiments, the silver microparticles are present at a concentration of 10-50 mg silver/cm² of film. In certain embodiments, the silver microparticles are present at a concentration of 10-25 mg silver/cm² of film. In certain embodiments, the silver microparticles have a mean diameter of less than 2 μm. In certain embodiments, the silver microparticles have a mean diameter ranging from 2 μm to 1,000 μm. In certain embodiments, the calcium is present in the film at a concentration of 0.00005-0.20 mg/cm² of film. In certain embodiments, the thrombin is present in the film at a concentration of at least 2 International Units (IU) thrombin per cm² of film. In certain embodiments, the thrombin concentration is greater than 2.5 IU thrombin per cm² of film. In certain embodiments, the thrombin concentration is less than 2,500 IU thrombin per cm² of film. In certain embodiments, the plasma is further mixed with cerium. In certain embodiments, the cerium is present in the polymerized gel at a concentration of 20-400 mM. In certain embodiments, the method further comprises adding an adhesive coating to the film. In certain embodiments, the adhesive coating is oxidized regenerated cellulose. In certain embodiments, the adhesive coating is present in the film at an amount of 5%-25% by weight. In certain embodiments, the method further comprises virally inactivating the human plasma. In certain embodiments, the method further comprises incubating the plasma at 37° C. after placing the plasma into the mold. In certain embodiments, the film has a thickness of less than 1 mm.

In certain aspects, described herein is a method of preventing a wound in a subject, comprising application of an effective amount of the dried film of any one of claims 1-29 to an area that is susceptible to forming a wound in the subject. In certain embodiments, the dried film is applied to the area that is susceptible to forming a wound less than 15 minutes after opening a storage container comprising the dried film. In certain embodiments, the dried film is applied to the area that is susceptible to forming a wound between 1 and 15 minutes after opening the storage container comprising the dried film. In certain embodiments, the wound is a non-healing wound. In certain embodiments, the wound is a hernia. In certain embodiments, the dried film degrades in less than 21 days after the application of the film to the area susceptible to forming a wound in the subject.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Production of Plasma Film Comprising Silver

Virally-inactivated (S/D treated and nanofiltered to remove viruses) human plasma (100 ml) was mixed with 1 ml CaCl₂ (2M) and 1 ml thrombin. The CaCl₂ was mixed gently and then poured into a 10×10 cm mold that was placed in an incubator at 37° C.×15 min. Silver (25 mg/ml plasma) was added either prior to the pressing step or after pressing via spray coating. A filter top was placed over the mold containing the solid (polymerized) gel before it was placed in a press fixture. The press fixture was put in a mechanical pressure and 1,000 PSI applied for 5-15 min. The film was removed from the press fixture and placed in biosafety cabinet at room temperature (25° C.)×4 hours before gamma sterilization. The sterilized film was removed from biosafety cabinet and stored in a sterile container at room temperature until use.

### Example 2: Application of Plasma Films Comprising Silver Microparticles for Prevention of Hernias

Virally-inactivated (S/D treated) human plasma (100 ml) is mixed with 1 ml CaCl₂ (2M) and 1 ml thrombin. The CaCl₂ is mixed gently and then poured into a 10×10 cm mold that is placed in an incubator at 37° C.×15 min. Silver microparticles (25 mg/ml plasma) is added either prior to the pressing step or after pressing via spray coating. A filter top is placed over the mold containing the solid (polymerized) gel before it is placed in a press fixture. The press fixture is put in a mechanical pressure and 1,000 PSI applied for 5-15 min. The film is removed from the press fixture and placed in biosafety cabinet at room temperature (25° C.)×4 hours before gamma sterilization. The sterilized film is removed from biosafety cabinet and stored in a sterile container at room temperature until use. A sufficient amount of film is applied to a subject in order to prevent a hernia. The film is effective for prevention of a hernia in the subject until the film degrades.

### Example 3: Plama Films Comprising Cerium for Treatment of Burn Wounds

Virally-inactivated (S/D treated) human plasma (100 ml) is mixed with 1 ml CaCl₂ (2M) and 1 ml thrombin (0.125 g thrombin in 50 mL sterile water). The CaCl₂ is mixed gently and then poured into a 10×10 cm mold that is placed in an incubator at 37° C.×15 min. Silver (25 mg/ml plasma) and cerium (20-400 mM/cm² of film) are added either prior to the pressing step or after pressing via spray coating. A filter top is placed over the mold containing the solid (polymerized) gel before it is placed in a press fixture. The press fixture is put in a mechanical pressure and 1,000 PSI applied for 5-15 min. The film is removed from the press fixture and placed in biosafety cabinet at room temperature (25° C.)×4 hours before gamma sterilization. A sufficient amount of film is applied to a subject with a burn wound. The film is effective in mitigation of the inflammatory injury resulting from the burn wound and enhancing wound healing of the burn compared to a subject with a comparable burn wound that has not been treated with the film.

### Example 4: Plasma Films Comprising an Adhesive Coating for Use in Soft Tissue Management

Virally-inactivated (S/D treated) human plasma (100 ml) is mixed with 1 ml CaCl₂ (2M) and 1 ml thrombin. The CaCl₂ is mixed gently and then poured into a 10×10 cm mold that is placed in an incubator at 37° C.×15 min. Silver (25 mg/ml plasma) is added either prior to the pressing step or after pressing via spray coating. A filter top is placed over the mold containing the solid (polymerized) gel before it is placed in a press fixture. The press fixture is put in a mechanical pressure and 1,000 PSI applied for 5-15 min. The film is removed from the press fixture and an adhesive coating of 5%-25% by weight comprising oxidized regenerated cellulose is applied to the film. The coated film is placed in biosafety cabinet at room temperature (25° C.)×4 hours before gamma sterilization. A sufficient amount of film is applied to soft tissue of a subject in need thereof. The coated film is effective for closing open tissue pockets thereby reducing the risks of seroma formation and other surgical site occurrences that can result from a surgical procedure the yields lipocutaneous flaps.

### Example 5: Films Consisting of Autologous Whole Blood for Application to Non-Healing Wounds

Virally-inactivated (S/D treated) human autologous whole blood is obtained from a human subject. The whole blood is then poured into a 10×10 cm mold that is placed in an incubator at 37° C.×15 min. A filter top is placed over the mold containing the solid (polymerized) gel before it is placed in a press fixture. The press fixture is put in a mechanical pressure and 1,000 PSI applied for 5-15 min. The film is removed from the press fixture, placed in biosafety cabinet at room temperature (25° C.)×4 hours before gamma sterilization. A sufficient amount of film is applied to the subject form which the whole blood was derived. The film is effective in promoting or enhancing the wound healing of a non-healing wound in the subject.

### Example 6: Production of Plasma Film Comprising Silver Microparticles Via Vacuum

Two methods of producing a plasma film comprising silver microparticles via vacuum were developed. The addition of silver microparticles while the plasma film is still wet was important for the abrasion-resistance of the silver in the final film. Abrasion resistance is important to ensure that the silver is not easily dislodged from the film during packaging and storage, or application of the film, for instance to a wound, hernia or burn.

Method 1

Virally-inactivated (Solvent/Detergent treated and nanofiltered to remove viruses, Octaplas, Octapharma AG; Lachen, Switzerland) human plasma (1,000 ml) was mixed with 0.86 g CaCl₂ and 0.21 g phosphatidylserine in a glass beaker and mixed with a stir bar for 2 min on medium speed. The plasma solution was then poured into a 30×30 cm mold and sprayed with metallic silver microparticles (2.5 to 25 mg/ml plasma, 15 μm size distribution, Technic Engineered Powders, Woonsocket, R.I. Product code: 81-800) using a powder coating sprayer (RaplixaSpray, Nordson Corporation, Westlake, Ohio). After silver application, the mold was placed in an incubator at 37° C. for 15 min to 60 min to allow polymerization (clotting) to form a gel. After clotting, another layer of silver was sprayed onto the polymerized gel. The polymerized gel was then put into a vacuum mold with a polyvinyl chloride (PVC) filter sheet placed on the bottom of the polymerized gel to act as a substrate for the final plasma film. The filter paper had either perforations or slits. In some production runs, a slitted paper was used with a single slit that is 0.02 inches wide and the full 30 cm length of the mold was used. In other productions runs, a filter paper with multiple 0.02 inch wide and 30 cm long slits spaced every 0.05 inches was used. A low vacuum pressure of 1.5 to 125 Torr was applied for 15 min to the polymerized gel to form the plasma film and remove the effluent from the gel. The effluent was collected in a bag for further use or to be discarded. After the film was formed via vacuum pressure, the 2% glycerin was added to the film. Two ways of adding the glycerin were developed. In the first method, the film was removed from the vacuum press and incubated in a 2% glycerin bath (glycerin in sterile water) for 15 minutes. In the second method, the film was left in the vacuum press and 200 mls of a 2% glycerin solution was added to the surface of the film. The vacuum was then turned back on to pull the glycerin over the film. The films were then dried for 30 min to 2 hours in an oven at 42° C. to 50° C. Drying the films at 50° C. was shown to accelerate the drying process without compromising the film strength. The finished film was abrasion resistant, see Example 8.

Method 2

Virally-inactivated (Solvent/Detergent treated and nanofiltered to remove viruses) human plasma (1,000 ml) was mixed with 0.86 g CaCl₂ and 0.21 g phosphatidylserine in a glass beaker and mixed with a stir bar for 2 min on medium speed. The plasma solution was then poured into a 30×30 cm mold and placed in an incubator at 37° C. for 15 min to allow polymerization (clotting) to form a gel. The polymerized gel was then put into a vacuum mold with a filter sheet made of polyvinyl chloride (PVC) placed on the bottom of the polymerized gel to act as a substrate for the final plasma film. The filter paper had either perforations or slits. In some production runs, a slitted paper was used with a single slit that is 0.02 inches wide and the full 30 cm length of the mold was used. In other productions runs, a filter paper with multiple 0.02 inch wide and 30 cm long slits spaced every 0.05 inches was used. A low vacuum pressure of 1.5 to 125 Torr was applied to the polymerized gel to form the plasma film and remove the effluent from the gel. The effluent was collected in a bag for further use or to be discarded. After the film was formed via vacuum pressure, it was incubated in a 2% glycerin bath for 15 min. The film was removed from the glycerin bath and one side immediate sprayed with metallic silver microparticles (2.5 to 25 mg/ml plasma, 15 μm size distribution) using a powder coating sprayer. The film was placed in a 37° C. oven for 5 min, removed, and the other side of the sprayed with the silver microparticles. The film was dried in the 37° C. oven for 1 hour. After the final drying step, the finished film was abrasion resistant, see Example 8.

The composition of an exemplary silver-microparticle containing film made by Method 1 with a 15 min glycerin bath soak and a 90 min drying step at 42° C. is shown in Table 1.

TABLE 1

| | | |
|---|---|---|
| Fibrin | 4.0 ± 1.0 | mg/cm² |
| Ag⁰ | 25 ± 5 | mg/cm² |
| Glycerin | 0.1 | mg/cm² |
| Water (moisture) | 0.9 | mg/cm² |

After production, the films can be sterilized with gamma irradiation (25 kGy, standard for medical products) and packed in Teflon pouches for storage.

### Example 7: Characterization of Physical Properties of Plasma Film Comprising Silver Microparticles

Silver content, protein composition, and excipient levels are determined via destructive analysis from films produced during the manufacturing process. Protein composition is determined from film samples subjected to digestion with trypsin and were analyzed on a high-resolution mass spectrometry analysis coupled with nanoflow ultra-performance liquid chromatography (UPLC).

Detailed protein and peptide analysis are identified and quantified. Silver concentration and distribution are quantified by x-ray fluorescence spectrometry of the film analyzing multiple sample areas. Glycerin concentration is measured by lab assay methods from a statistically representative number of film samples.

Endotoxin

Endotoxin is determined by USP <85> BACTERIAL ENDOTOXINS TEST.

Sterility

Sterility is determined by USP <71> STERILITY TESTS.

Silver Content

Content and distribution of Ag⁰ is determined using a handheld x-ray fluorescence (XRF) spectrometer (X-550, SCiAps, Inc., Woburn, Mass.). One hundred (100) 1 cm×1 cm squares of plasma film comprising silver microparticles is obtained from a randomly selected 10 cm×10 cm film for testing. Each 1 cm² sample is placed on a sterile surface. The analyzer is turned on, an analysis mode selected, and a measurement obtained as per manufacturer's instructions. Each measurement takes a few seconds with the data recorded wirelessly on a laptop computer with cloud-based storage and backup.

Fibrin Content

Content of fibrin is determined by proteomic analysis using an Ultimate 3000 nano ultra-high performance liquid chromatography (UHPLC) system coupled with a Q Exactive HF mass spectrometer (Thermo Fisher Scientific, US) with an ESI nanospray source.

The sample protein pellets are dissolved in 50 mM ammonium bicarbonate. The solution is transferred into Microcon devices YM-10 (Millipore) and centrifuged at 12,000×g at 4° C. for 10 min. 200 μL of 50 mM ammonium bicarbonate is added to the concentrate followed by centrifugation and repeated once. The sample is reduced by 10 mM dithiothreitol (DTT) addition at 56° C. for 1 hour and alkylated by adding 20 mM indole-3-acetic acid (IAA) at room temperature in the dark for 1 hour. The device is centrifuged at 12,000×g at 4° C. for 10 min and washed once with 50 mM ammonium bicarbonate. 100 μL of 50 mM ammonium bicarbonate and 0.25% free trypsin are added into the protein solution at a ratio of 1:50 and the solution is incubated at 37° C. overnight. Finally, the device is centrifuged at 12,000×g at 4° C. for 10 min. 100 μL of 50 mM ammonium bicarbonate is added into the device and centrifuged, and then repeated once. The extracted peptides are then lyophilized to near dryness, and resuspended in 10 μL of 0.1% formic acid for liquid chromatography tandem mass spectrometry (LC-MS/MS) analysis. Columns and buffers. Nanocolumn trapping column (PepMap C18, 100 Å, 100 μm×2 cm, 5 μm) and an analytic xl column (PepMap C18, 100 Å, 75 μm×50 cm, 2 μm). Loaded sample volume is 1 μg; mobile phase A: 0.1% formic acid in water; B: 0.1% formic acid in 80% acetonitrile. Total flow rate: 250 nL/min. LC linear gradient: from 2 to 8% buffer B in 3 min, from 8% to 20% buffer B in 50 min, from 20% to 40% buffer B in 43 min, then from 40% to 90% buffer B in 4 min.

Antimicrobial Potency

Potency is determined by the zone of inhibition (ZOI) Test. This test demonstrates the antibacterial efficacy of the silver microparticle-containing film through the elution of a low level of silver (Ag+) ions from the product's silver microparticles. For example, 25 mg/cm² concentration of silver plus fibrin should produce a≤1.5 mm ZOI for both gram positive and gram-negative bacteria.

The following standard bacterial strains are used: Staphylococcus aureus (methicillin-resistant, (mr) ATCC #43300), Staphylococcus aureus (methicillin-sensitive, (ms) ATCC #25923), Escherichia coli (ATCC #35218), Pseudomonas aeruginosa (ATCC#27853), and Staphylococcus epidermidis (ATCC #35984). Petri dishes (100 mm×15 mm) containing Luria-Bertani (LB) nutrient agar are streaked with the different bacteria cultures. Three (3) samples of the silver microparticle-containing film (1-cm disc) are placed onto the nutrient agar. Gentamicin- (2 μg/disc) and saline treated (50 μl) filter paper (1-cm) discs serve as positive and negative controls, respectively. Plates are incubated at 36° C. for 24 h for optimal bacterial growth, then photographed and the diameters of the ZOI of the discs measured using ImageJ (National Institutes of Health [NIH]). The antibacterial activity is expressed as the mean of the diameter of the ZOI (mm) minus the diameter of the discs (±standard error of the mean [SEM]). Statistical analyses are performed using a 1-way analysis of variance (ANOVA).

Process Residuals

Process residuals from the plasma, such as sodium citrate dihydrate, sodium dihydrogen-phosphate dihydrate, and glycine are determined by a combination of mass spectrometry and biochemical analyses.

### Example 8: Characterization of the Mechanical Properties and Stability of Plasma Films Comprising Silver Microparticles

Materials and Methods

The film was prepared, packaged and sterilized as described in Example 6. Briefly, 100 mL of Octaplas plasma (Ocatapharma AG) was placed in a mold and recalcified with 1.0 ml of 2M CaCl₂ and allowed to clot for 15 minutes at 37° C. After 15 minutes the mold was attached to the vacuum pump and low vacuum of between 1.25 mm Hg and 125 mm Hg was applied for 5 minutes. The gel was soaked in 5% glycerin for 15 minutes and then coated with silver microparticles at a concentration of 25 mg/cm², and dried to a film. The films were packaged in a foil pouch and gamma sterilized at 20 kGy and stored at room temperature for 15 months.

Burst Pressure

Burst pressure was measured according to the Standard Test Method for Burst Strength of Surgical Sealants (ASTM-F 2392-04). A film was fastened in a fixture according to ASTM-F 2392-04 and the fixture was connected to a pump with a pressure transducer being attached inline between the pump and the burst pressure fixture. Pumping a fluid into the system increased the pressure until the film bursts. The burst pressure is indicated as [mm Hg].

Fold Number and Fold Endurance

The fold number and fold endurance were also determined for the silver microparticle-containing films.

Fold number is the determination of the number of times a film can be folded to determine the film flexibility. Such determination is accomplished by folding one half of the film on top of the other half, turning the stack by 90° and again folding one half of the stack on top of the other and repeating until the film breaks or rupture. Each folding without triggering film rupture increases the fold number by 1. This test is particularly useful for the assessment of a plasma-based film's ability to maintain its integrity when being bent in tight turns.

Fold endurance is the determination of the number of times a film can be folded and unfolded and is also a marker of the film flexibility. Such determination is accomplished by repeatedly folding one half of the film on top of the other half and unfolding to its original position. Fold endurance is expressed by the number of such folding/unfolding repeats and provides a means for wear resistance estimation.

Stability

Multiple silver microparticle coated plasma films were prepared. Five films were tested for burst pressure, fold number, endurance number and film thickness after preparation. After 464 days (approximately 15 months) of storage at room temperature five additional films were re-evaluated for fold number, endurance number and film thickness.

Abrasion

A 5 cm×5 cm silver coated film is weighed, the brushed 100 times on both sides of the film using a 2 inch wide paint brush. The film is weighed again post 100 brush sequences.

Results

The mechanical properties of the newly synthesized films are shown in Table 2.

TABLE 2

| | Thickness | Burst Pressure | Fold | Fold |
|---|---|---|---|---|
| Film | [mm] | [mmHg] | number | Endurance |
| 1 | 0.03 | 941 | 5 | 100 |
| 2 | 0.03 | 878 | 5 | 100 |
| 3 | 0.03 | 894 | 5 | 100 |
| 4 | 0.03 | 1009 | 5 | 100 |
| 5 | 0.03 | 992 | 5 | 100 |
| Average | 0.03 | 942.8 | 5 | 100 |

The mechanical properties of the films stored at room temperature for 15 months are shown in Table 3.

TABLE 3

| | Thickness | Burst Pressure | | Fold |
|---|---|---|---|---|
| Film | [mm] | [mmHg] | Fold number | Endurance |
| 1 | 0.03 | 908 | 5 | 100 |
| 2 | 0.03 | 915 | 5 | 100 |
| 3 | 0.03 | 972 | 5 | 100 |
| 4 | 0.03 | 941 | 5 | 100 |
| 5 | 0.03 | 949 | 5 | 100 |
| Average | 0.03 | 937 | 5 | 100 |

As shown in Tables 2 and 3, the storage of the film for 15 months at room temperature did not decrease the films' flexibility, as indicated by the same fold number and fold endurance metrics: fold number of 5 and fold endurance of 100 for both newly synthesized films and films stored for 15 months. In addition, the burst pressure of the film was not significantly reduced. In fact the average burst pressure decreased by only 5 mm Hg. The films stored for 15 months had a burst pressure 99.5% the same as the newly synthesized films.

The abrasion resistance of the films are shown in Table 4.

TABLE 4

| | Weight Pre | Weight Post |
|---|---|---|
| Film | brush grams | brush grams |
| 1 | 1.100 | 1.109 |
| 2 | 1.094 | 1.103 |
| 3 | 1.109 | 0.993 |
| 4 | 1.107 | 1.107 |
| 5 | 1.137 | 1.130 |
| Average | 1.106 | 1.122 |

As shown in Table 4, the silver microparticle-containing films did not lose a significant amount of weight after being brushed 100 times on both sides, and thus the silver embedded in the films is abrasion resistant.

Abrasion resistance of the silver microparticles from the film is an important characteristic. An abrasion resistant film does not have silver dislodging from the film and coming into contact with tissue that is not intended to be treated with the silver/plasma film. The silver abrasion resistant film also allow for accurate dose determination of the film, and the silver microparticles do not dislodge from the film during packaging, sterilization or storage. Applying the silver by spray coating to a wet film prevents the silver from dislodging from the surface after subsequent drying. Without wishing to be bound by theory, applying the silver microparticles by spray coating to a wet film results in the silver microparticles becoming physically entrapped into the fibrin surface of the film and do not abrade with touch, handling, sterilization or packaging.

Additional experiments were conducted to assess the fold endurance of a newly synthesized fibrin film containing silver microparticles. Three 5 cm×5 cm films were tested for fold endurance of 1000 folding and unfolding repeats according to the fold endurance assay described. The assay was halted at 1000 folds, but the film had not failed by that point and the assay could have continued. Thus, the films have a fold endurance of at least over 1000. Burst pressure was also measured from a 5 cm×cm film made in the same film production run and is shown in Table 5. As shown in Table 5, the folding endurance assay did not significantly decrease the burst pressure of the fibrin film containing silver microparticles.

TABLE 5

| | Pre folding burst | Post folding burst |
|---|---|---|
| Film | mmHg | mmHg |
| 1 | 907 | 943 |
| 2 | 986 | 905 |
| 3 | 1074 | 988 |
| Average | 989 | 945.3 |

### Example 9: In Vitro Ionization of Silver Microparticles

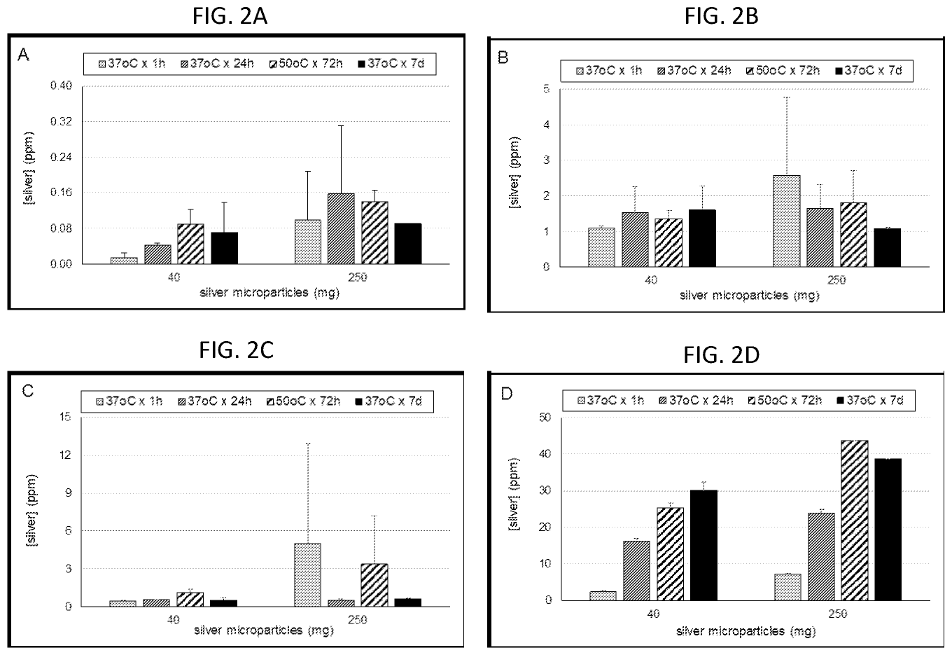

The elution of silver ions from silver microparticles in various liquid vehicles was investigated according to a published protocol (Sussman, Assessment of total silver and silver nanoparticle extraction from medical devices. Food Chem Toxicol 2015; 85:10-19). Four solution vehicles were used: (1) distilled, deionized water (FIG. 2A), (2) normal (0.9%) saline (FIG. 2C), (3) human plasma (FIG. 2D), and (4) simulated body fluid (SBF) (FIG. 2B). Silver microparticles were weighed to a final concentration of 40 mg/mL or 250 mg/mL in 10 mL of sterile vehicle, placed in 20 mL HDPE scintillation vials with polyethylene cone lid inserts and then agitated in temperature-controlled shakers for 1 hour at 37° C., 24 hours at 37° C., 72 hours at 50° C. and 7 days at 37° C. After incubation, the vehicle was removed from the vials, placed into 15 mL polystyrene centrifuge tubes, aliquots diluted 1:100 in 1.2 N HCl, and analyzed for silver content by inductively coupled plasma mass spectrometry (ICP-MS). All samples were run in triplicate.

Metallic silver microparticles were effectively insoluble, especially in water, SBF and saline where the highest concentrations of soluble silver (Ag+) measured were approximately 0.2 ppm, 2.6 ppm, and 5 ppm, respectively (FIG. 2A-D). Equilibrium was achieved between the silver ions and salts present in saline and SBF (FIGS. 2C and 2B); this equilibrium was achieved at a greater than ten-fold higher concentration of Ag+ than when the microparticles were incubated in deionized water (FIG. 2A). Silver was more soluble in plasma yielding a maximum concentration of 44 ppm (FIG. 2D). Without wishing to be bound by theory, organics such as proteins shift the equilibrium of soluble silver since Ag+ binds strongly to plasma albumins and macroglobulins. However, even under optimal elution conditions, <0.02% of the total silver metal incubated dissolved. Furthermore, incubating a higher concentration of metallic silver did not result in a predictable increase in soluble Ag+. While incubating silver microparticles at 250 mg/mL generally yielded a higher concentration of silver ions than a concentration of 40 mg/mL, regardless of the vehicle or incubation conditions, the relative increase in soluble Ag+ was approximately +50% to +75% despite the >600% difference between the starting concentrations of silver metal.

In summary, metallic silver is widely accepted to be biologically inert and thus poorly absorbed into the body via inhalation, ingestion, or cutaneous contact. The preceding data further underscore the inert, insoluble nature of metallic silver and thus predict very low levels of systemic absorption in vivo.

### Example 10: In Vivo Tissue Distribution of Silver

Although metallic silver is inert and as such less toxic than silver salts like silver nitrate (AgNO₃) and silver sulfadiazine (SSD), in the presence of body fluids it ionizes, releasing the biologically active Ag+ that shows a strong affinity for sulfhydryl groups and other anionic ligands of proteins, amino acids and cell membranes. And, despite evidence from many experimental and clinical studies indicating that silver is not retained in any organ of the body (except rarely in the skin with cases of argyria), the amount of silver detected in selected rat organs and plasma following the subcutaneous implantation of a fibrin film containing metallic silver microparticles (15 μm; Technic, Inc.) after 28 days is assessed.

Methods

Mouse

Incisional hernias are made in an equal number of male and female rats (n=12/sex/group) and are randomly assigned to a treatment (fibrin film containing 2.5 mg/cm² and 25 mg/cm² metallic silver microparticles, 15 μm) versus control group under GLP. At 28 days, necropsies are performed by an independent, blinded veterinary pathologist. Plasma and tissue samples are collected for gross and histopathology, complete blood count and clinical chemistry, and silver content determination via ICP-MS. The detection limit for silver ranges from 0.003-0.018 parts per billion (ppb) with measurements below this level recorded as 0.0. Whole blood is collected from fasted animals, mixed with heparin to derive plasma for clinical chemistry, hematology and coagulation assays.

Porcine

The distribution of silver following the application of fibrin film containing 2.5 mg/cm² and 25 mg/cm² metallic silver microparticles, 15 μm, is also characterized in a porcine model of incisional hernia prevention. In brief, a 10-cm full-thickness laparotomy incision is made through the midline fascia. The intestines are briefly manipulated before closing the fascial incision with five interrupted, absorbable 2-0 BIOSYN™ sutures (Covidien, Mansfield, Mass.) placed 0.5 cm from either end of the laparotomy incision and 1.5 cm apart. A total of 6 animals (Yorkshire pigs, female, 90-105 kg) are randomized to receive either fibrin film containing 2.5 mg/cm² metallic silver microparticles, 15μ, fibrin film containing 25 mg/cm² metallic silver microparticles, 15μ, or an saline topically applied to the sutured myofascial incisions before skin closure. The total surface area treated is a 2-cm area surrounding the 10-cm long fascial incision. The skin flap is then closed in two-layers. After 30-60 min of recovery under heat lamps, the pigs are returned to fresh individual pens.

On Day 28 or 90, selected animals are euthanized, a necropsy is performed, and multiple organs weighed, washed in saline and multiple 1-gram samples from the stomach, small intestines, large intestines, heart, liver, kidneys, lungs, skeletal muscle, spleen, pancreas, adrenals, testes, adipose tissue, brain and skin are harvested, placed in cryovials and frozen in liquid nitrogen for subsequent analysis. The silver content of each tissue is measured via ICP-MS.

Results

Mouse

Little to no evidence of biochemical organ injury or gross organ pathology is observed in the mouse model. Wound sites in animals treated with fibrin film containing 2.5 mg/cm² and 25 mg/cm² metallic silver microparticles are associated with an infiltrate of macrophages and multinucleated giant cells. No necrosis is noted at the implant site in any of the treated or control animals.

Little to no behavioral, clinical, gross anatomic or histopathologic evidence of disease involving the hepatic, musculoskeletal, renal or hematologic systems (i.e., bleeding) is observed.

Porcine

Silver is not detected in the small intestines, large intestines, pancreas, adrenals, testes, adipose tissue, liver, or skin. The animals exhibit normal growth and weight gain for the duration of the study. At necropsy, there is no evidence of clinical or histopathology involving any organ, including the liver, spleen, kidney or skin.

### Example 11: In Vivo Wound Treatment with a Silver Microparticle-Containing Plasma Film

Fibrin is central to tissue repair as the polymer network it forms in the wound acts not only as a hemostatic barrier but also a protein scaffold that supports migrating immune cells and sequesters soluble signaling molecules. However, the physiological properties of fibrin are complex and their relation to fibrin's macromolecular structure is incompletely understood. See, e.g., Litvinov R I, Weisel J W. Fibrin mechanical properties and their structural origins. Matrix Biol 2017; 60-61:110-23. Thus, the efficacy of silver microparticles plus fibrin on wound healing in diabetic mice when the source of the fibrin was a liquid tissue sealant versus a dried planar plasma film was assessed.

Materials and Methods

Animals. All procedures were performed with the prior approval of LabCore's Institutional Animal Care and Use Committee. Genetically diabetic C57BL/KsJ-db/db (male, 14-16 weeks) mice were obtained from Jackson Laboratories (Bar Harbor, Me.). The animals were acclimated to laboratory conditions for a minimum of 2 days prior to undergoing surgery and all were provided access to water and standard rat chow ad libitum. These leptin receptor-deficient animals are a widely accepted model of Type 2 diabetes that allowed comparison of changes in wound healing with and without treatment. See, e.g., Kleinert M, Clemmensen C, Hofmann S M, et al. Animal models of obesity and diabetes mellitus. Nat Rev Endocrinol 2018; 14:140-62

Excisional Wounding. To examine the effect of silver plus either a liquid fibrin sealant or when incorporated in a dry plasma film on diabetic wound healing, mice were placed under isoflurane anesthesia and their dorsum shaved with electrical clippers. One hundred microliters (100 μl) of 1/30 dilution of 0.3 mg/ml buprenorphine was injected subcutaneously, then the dorsum prepared with betadine antiseptic solution and alcohol. Circular excisional wounds measuring 2-cm in diameter were made on the back of each mouse using a stencil and scissors, including removal of the panniculus carnosus layer. The animal was then randomly assigned to treatment with either liquid fibrin sealant with silver or dry plasma film with silver versus a saline control. The control mice had 250 μl of saline applied to the open wounds while the experimental mice were treated with either liquid fibrin sealant [300 μl of liquid fibrin sealant (TISSEEL Baxter Healthcare Corp, Hayward, Calif.) plus 6 mg/cm² silver microparticles (15 μm, Technic, Inc., Woonsocket, R.I.)] or dry plasma film with silver (2-cm discs of plasma film containing 10 or 25 mg/cm² silver microparticles). The wounds were subsequently covered with an occlusive dressing (Tegaderm™, 3M, St. Paul, Minn.) secured with sutures to prevent the animals from removing the dressings, thus keeping the wounds clean and moist. Furthermore, this is analogous to the management of diabetic ulcers in patients. The mice were then returned to individual cages and allowed to awaken and resume normal activity. The mice were examined, and photographs taken of the dorsal wounds at weekly intervals starting on Day 0. Twenty-eight (28) days after treatment, the mice were euthanized. Serial wound photographs were analyzed using a standard imaging program, Image J (NIH).

Wound Healing Evaluations. Each animal's weekly wound photographs were measured and serial photographs compared to determine the wound closure rate [(baseline area—detected area)/baseline area×100%]. The area under each wound healing curve (AUC) was determined using the trapezoidal rule.

Statistics. Statistical analysis was performed comparing the mean AUC values from control and experimental groups using Student's t-test with significance at P<0.05.

Results

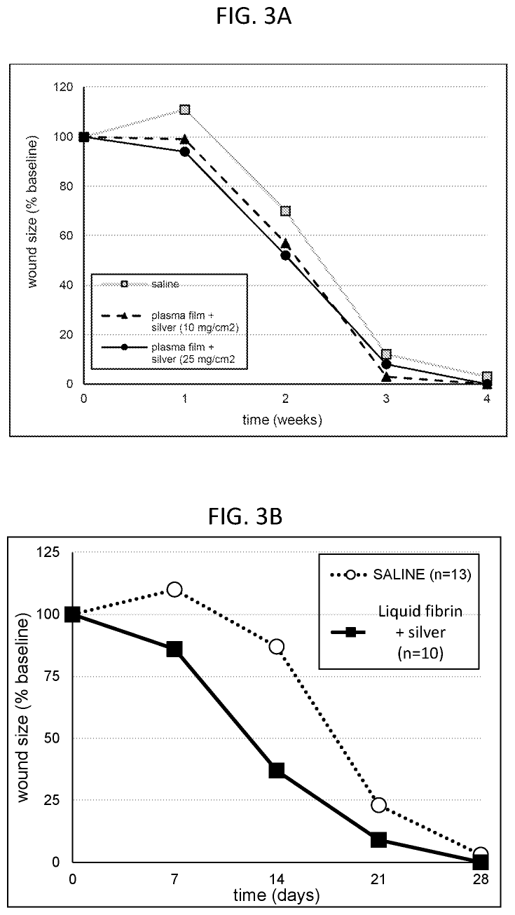

At the end of 28 days, most of the dorsal wounds had healed irrespective of treatment (FIGS. 3A and 3B). However, the mice treated with either liquid fibrin sealant and silver microparticles or dry plasma film with silver microparticles healed their wounds more rapidly than the saline-treated controls as evidenced by smaller areas under the wound healing curves (182±29 vs. 271±37, p<0.001 and 205±28 vs. 287±74, p=0.04, respectively).

Wounds treated with silver plus fibrin healed more quickly, completely re-epithelializing within 28 days as compared to saline alone. FIG. 3A shows the wound area after treatment with the silver microparticle-containing film. FIG. 3B shows the wound area after treatment with the silver microparticles+liquid fibrin. Mean area under the curve (AUC) of the wound healing time was determined for the wounds treated with silver-containing film and silver+liquid fibrin. Table 6 shows the AUC for the silver microparticle-containing film, Table 6 shows the AUC for the silver microparticle+liquid fibrin.

TABLE 6
AUC of wounds treated with silver microparticle-containing film

| | | | P value |
|---|---|---|---|
| treatment | mean AUC | SD | (vs. control) |
| SALINE (control) n = 5 | 244 | 38 | — |
| silver microparticle-containing | 205 | 28 | 0.04 |
| film (25 mg/cm²) n = 10 | | | |
| silver microparticle-containing | 209 | 22 | 0.06 |
| film (10 mg/cm²) n = 7 | | | |

TABLE 7
AUC of wounds treated with silver microparticles and liquid fibrin

| | | | P value |
|---|---|---|---|
| treatment | mean AUC | SD | (vs. control) |
| SALINE (control) | 271 | 37 | — |
| Silver microparticles plus | 182 | 29 | <0.001 |
| liquid fibrin | | | |

Data are mean±standard deviation (SD)

Due to the diabetic mouse model, the silver treatment did not restore wound healing to normal. However, both films containing silver microparticles (10 mg/cm² and 25 mg/cm²) resulted in increased healing time as compared to saline (mean AUC of 205 and 209 for the silver containing films as compared to 244 for saline). Unexpectedly, the dried films containing silver microparticles were also as effective at healing wounds as the liquid fibrin plus silver (mean AUC of 182). Remarkably, the degree of accelerated wound healing observed in the treated animals was virtually identical comparing the two formulations tested. The dry planar fibrin in the plasma film was as effective compared to the liquid fibrin. This was an unexpected finding since the hydration state and tertiary structure of fibrin in the plasma film is significantly altered due to the drying and compression during production when compared to the structure of fibrin in liquid form. A complex fibrous protein like fibrin would not be expected to maintain its biological potency and effects on wound healing after such physiochemical changes. See, e.g., Weisel J W, Litvinov R I. Fibrin Formation, Structure and Properties. Subcell Biochem 2017; 82:405-56.

The level of improvement observed with the dry film and silver rivaled that demonstrated by various drugs, growth factors and stem cell therapies. See, e.g., Brown R L, Breeden M P, Greenhalgh D G. PDGF and TGF-alpha act synergistically to improve wound healing in the genetically diabetic mouse. J Surg Res 1994; 56:562-70; Sun Y, Song L, Zhang Y, Wang H, Dong X. Adipose stem cells from type 2 diabetic mice exhibit therapeutic potential in wound healing. Stem Cell Res Ther 2020; 11:298; and Wang F, Zhang C, Dai L, et al. Bafilomycin A1 Accelerates Chronic Refractory Wound Healing in db/db Mice. Biomed Res Int 2020; 2020:6265701. Notably, the accelerated healing in the silver-treated animals was most pronounced in the first 14 days following treatment. This observation was consistent with the activation of macrophages as these cells are critical during the early phase of wound healing.

In summary, silver microparticles combined with dried fibrin (plasma film) unexpectedly accelerated wound healing in genetically diabetic mice.

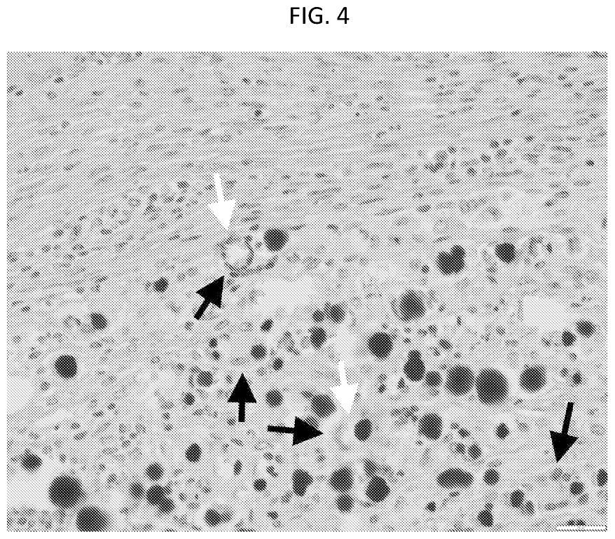

FIG. 4 shows a representative histology sample of healing excisional skin wound in a diabetic mouse (C57BL/KsJ-db/db) treated with the dried fibrin film containing silver microparticles. Several multi-nucleated giant cells can be seen surrounding silver microparticles (black arrows) and surrounding air spaces resulting from dislodgement of silver microparticles during tissue processing (white arrows). 40× magnification. Without wishing to be bound by theory, it is believed that multinucleated foreign body giant cells are important for wound healing.

### Example 12: Degradation of Silver Microparticle-Containing Films In Vivo

Materials

Incision

Sprague-Dawley rats (male, 250-300 g, Charles River, Cambridge, Mass.) are placed under isoflurane anesthesia; the ventral abdominal wall hair is shaved with electric clippers, and the surgical field is prepared with 70% alcohol. A 6 cm×3 cm, rectangular, full-thickness skin flap based 2 cm lateral to the ventral midline is raised through the avascular prefascial plane, thereby separating the skin incision from the underlying fascial wound-healing environment. The 1:2 ratio of flap length to width is maintained to prevent ischemia of the skin flap. A 5-cm midline laparotomy incision is made, the intestines are manipulated, and then the myofascial incision is closed with two interrupted 5-0 plain catgut (rapidly absorbable) sutures placed 5 mm from the cut myofascial edges and one-third the distance from the cranial and caudal ends of the midline laparotomy incision, respectively, before the skin flap is closed with a continuous 4-0 vicryl suture to prevent intestinal evisceration. Immediately after the surgery, 0.4 mL of bupivacaine 0.25% is infused subcutaneously around the abdominal incision, and the rats are observed every 2 min until awake and resuming normal activity. Thereafter, the rats are returned to individual cages and monitored twice daily. At 12 and 18 h after operation, 0.05 mg/kg of buprenorphine is injected subcutaneously.

Silver Microparticle-Containing Film Application

A total of 82 rats are randomly placed into three control group (saline alone, dried fibrin film alone, and liquid fibrin sealant with silver microparticles, as described in WO2013138238), and two experimental groups (dried fibrin films containing 2.5 mg/cm² and 25 mg/cm² of silver microparticles (150). Animals in the have the selected treatment applied to their sutured myofascial incisions before skin closure. On days 21 and 28, all animals are inspected for the presence of an obvious abdominal protrusion (clinical hernia) and recorded as such. The animals are then euthanized, have their abdominal wall excised, and overlying skin carefully separated from the myofascial surface and hernia sac if present. Each specimen is carefully examined, and any gap between recti is measured. If a gap is found to be greater than 2 mm in diameter, it is classified as an anatomical hernia. The total area of the myofascial defect is also determined by measuring the maximal transverse and cranial caudal dimensions, and then calculated using the equation for an ellipse (π×r₁×r₂; r₁=½ the transverse diameter and r₂=½ the craniocaudal diameter). Incisional hernia incidence and size, tensile strength, and tissue histology are assessed after 21 and/or 28 days. Histology of the abdominal wall muscle and surrounding collagen is assessed via hematoxylin-eosin and Sirius Red staining. In addition, slides are scored for giant cells, fibrosis, fibrotic density, and angiogenesis according to the method described in Hansen et al, Am J Pathol 2003; 163:2421-2431. Inflammation is evaluated by counting the number of common inflammatory cells according to the method described in Mace et al, Wound Repair Regen 2007; 15:636-645.

Tensile Strength Assay

A further study in rats is done to determine that the tensile strength of a normally healing abdominal incision is not affected by treatment using silver microparticle containing films. An established protocol for creating incisional hernias is used, (Dubay, Ann Surg 2004; 240:179-186) except the 5-cm midline laparotomy incision is closed using a continuous running 4-0 vicryl suture rather than 2 interrupted 5-0 plain catgut sutures. The continuous closure using a slowly dissolving suture material rather than a rapidly dissolving one allows an adequate amount of time for the myofascial wound to heal normally (analogous to the standard of care in patients) and thus avoid the formation of an incisional hernia. The animals are randomly placed into two control groups (saline alone and fibrin film alone), and two experimental groups (2.5 mg/cm² versus 25 mg/cm² of silver microparticle containing fibrin films). On Day 21 or 28, all animals are euthanized, the entire ventral abdominal wall is excised and two strips of muscle are collected for mechanical testing. Tensiometric analysis of the abdominal wall muscle strips is performed within 6 h of necropsy. Stretch loading is used to facilitate mechanical characterization of the fascia-fascia interface using an Instron Tensiometer (model MicroTester®). Force and tissue deformation data is simultaneously captured via computer and data analysis performed using Bluehill® Software. Failure of the specimen is defined at the yield point, rather than at the point of ultimate tissue disruption.

Results

No gaps are observed in mice treated with the dried fibrin films containing 2.5 mg/cm² and 25 mg/cm² of silver microparticles on day 21 or 28.

A reduction of both incisional hernia incidence and hernia size is observed between the saline control group and the mice treated with fibrin films containing silver microparticles. Histological samples show an increase in new fibrosis in the treated animals as compared with the saline control. After 21 or 28 days, the tensile strength of the myofascial incisions is the same regardless of treatment group.

### Example 13: Human Trial for Diabetic Foot Ulcers (DFUs)

Diabetic foot ulcers are a common, complex and serious complication of diabetes, one that is associated with significant morbidity, mortality and healthcare costs. DFUs annually affect some 26 million people worldwide (Armstrong, N Engl J Med 2017; 376:2367-2375), at a cost of over $13 billion in the US alone (Rice, Diabetes Care 2014; 37:651-658), and with a five-year mortality rate equivalent to that for esophageal, liver and lung cancer (Singh, JAMA 2005; 293:217-228). But, perhaps the most feared consequence of developing a DFU is the risk of amputation. In 2016, there were over 130,000 lower-limb amputations in people with diabetes which translates into 1 amputation every 4 minutes (CDC, National Diabetes Statistics Report, 2020). An ulcer preceded 85% of these amputations (Singh, JAMA 2005; 293:217-228; Driver, J Am Podiatr Med Assoc 2010; 100:335-341). Unfortunately, current treatments for DFUs have one overarching limitation. Treatments either decrease bacterial growth which hinders cellular regeneration, or they enhance cellular growth but have limited antibacterial activity. No currently available treatment effectively enhances cellular growth while inhibiting bacteria. Since DFUs commonly have both an increased microbial burden (bioburden) while simultaneously lacking adequate regenerative cellular and growth factor responses (inadequate healing), optimal treatment must simultaneously address the wound bioburden while also restoring the wound's regenerative capacity in a safe and non-toxic manner. Fibrin films containing silver microparticles leverage the medicinal properties of silver to significantly enhance wound healing while controlling the local bioburden.

Study Design

Multicenter, parallel group, standard of care controlled, phase 1 study of thirty (30) adult patients undergoing treatment for a diabetic foot ulcer. Participants are equally randomized to the study arms. Treatment involves the weekly application of a silver microparticle-containing film (15 μm particles at 2.5 to 25 mg/cm² film) until the wound heals, not to exceed 12 successive weeks versus standard-of-care. At the baseline visit (Day 1), all wounds are inspected, debrided and/or cleansed before the silver microparticle-containing film is applied topically to cover the entire surface area of the wound followed by placement of a dressing for 7±2 days. The dressing is only removed at weekly visits, except for a dressing change on Day 3 (±1 day) to investigate potential hypersensitivity reactions and at the discretion of the Investigator to manage excessive wound exudate. Additional debridement is performed at subsequent clinic visits to remove any necrotic or unwanted tissue as deemed necessary by the Investigator. The patient's diabetic foot ulcer is monitored on a weekly basis, including measurements of size and depth along with assessment of ulcer deterioration, pain or signs of infection.

Duration

Study duration is up to a total of 26 weeks from enrollment to end of study. This includes a two-week active run-in followed by up to 12 weeks of randomized treatment, a follow up visit 2 weeks after complete closure of a participant's wound completely, plus a final follow-up visit 12 weeks after the last treatment.

Efficacy Assessments

The primary endpoint for the clinical trial is the safety of topically applying a plasma film containing metallic silver microparticles in patients with non-healing diabetic foot ulcers.

Secondary endpoints will include:
Percent of study wounds healed during the post-treatment weeks 1 through 12
Time to complete wound closure
Percent of area (cm²) reduction during post-treatment weeks 1 through 12
Ulcer recurrence rate 3 months after treatment
Wound infection rate during study period
Pain (VAS)
Quality of life (Wound-QoL, SF-36) and
Cost of treatment

Safety Assessments

Safety assessments include adverse events (AEs), serious adverse events (SAEs), physical examinations, vital sign measurements, clinical laboratory evaluations, and reasons for treatment discontinuation due to toxicity.

Study Population

Adult patients with diabetic foot ulcers located distal to the malleolus with controlled diabetes mellitus and without significantly compromised arterial circulation.

Inclusion Criteria
1. Men or women 18-75 years of age.
2. The subject is able and willing to adhere to study procedures and informed consent is obtained.
3. A non-healing ulcer that is diabetic in origin, located on the foot as defined by beginning below the malleoli of the ankle, and of >30 days duration prior to the day of screening that has not adequately responded to conventional wound healing therapy.
4. Target ulcer surface area between 1-10 cm² after debridement with no active infection. Sharp debridement is done prior to randomization. Subject's informed consent for participating in this study must be obtained prior to proceeding with sharp debridement.
5. Patients with Type 1 or Type 2 diabetes (criteria for the diagnosis of diabetes mellitus per American Diabetes Association).
6. Additional wounds may be present but not within 3 cm of the target ulcer.
7. Patient has adequate arterial perfusion of the affected extremity as demonstrated by any one of the following within the past 90 days:toe pressure (plethysmography)>50 mm/Hg OR Systolic blood pressure ABI with results≥0.70 and ≤1.2 OR TcPO2≥30 mm Hg from the foot OR Doppler arterial waveforms consistent with adequate flow in the foot (biphasic or triphasic waveforms at the ankle of affected leg).
8. Target ulcer involves a full thickness skin loss, but WITHOUT exposure of tendon, muscle, or bone that has been present≥4 weeks.
9. HbA1c<12% taken within 90 days prior to randomization.
10. Serum creatinine<3.0 mg/dL within the last 6 months.
11. Willing and able (subject or responsible caregiver) to maintain required off-loading (as applicable for the location of the ulcer).
12. Negative pregnancy test
13. Use of reliable method(s) of contraception and/or abstinence, for the duration of the treatment product exposure for woman of childbearing potential.

Exclusion Criteria
1. Suspected or confirmed signs/symptoms of wound infection (prior to enrollment the infection may be treated, and subject reconsidered for study participation).
2. Patients presenting with an ulcer probing to bone (University of Texas Grade IIIA-D). A positive probe-to-bone is confirmed when bone or joint can be felt with a sterile, ophthalmological probe.
3. Hypersensitivity to silver.
4. Hypersensitivity to FFP or to plasma-derived products including any plasma protein.
5. The subject was previously entered into this study or had participated in any study drug or medical device study within 30 days of enrollment.
6. Currently on a treatment regimen or medications which in the opinion of the Investigator are known to interfere with wound healing (for example: cancer chemotherapy or equivalent immunosuppressants, systemic steroids>10 days of treatment, cytostatic drugs, certain allergy medications, COX-2 inhibitors, or radiation therapy).
7. Excessive lymphedema that in the opinion of the Investigator will interfere with wound healing.
8. Any condition interfering with subject's ability to comply with the treatment regimen.
9. Active Charcot foot or Charcot deformity with boney prominence that in the opinion of the Investigator will inhibit wound healing.
10. Wounds secondary to vasculitis, neoplasms, or hematological disorders. Patients on anticoagulation medication will as in any surgical procedure, be monitored according to the protocols employed at the enrolling center.
11. A condition, other than diabetes which, in the opinion of the Investigator, would compromise the safety of the subject or the quality of the data or seriously interfere negatively with the normal parameters of the wound healing process.
12. Subjects on dialysis.
13. History of radiation to the foot.
14. Ulcers present for >18 months.
15. Clinically relevant history of alcohol or drug use disorder.
16. Patients with uncontrolled autoimmune connective tissue diseases.
17. Patients who are pregnant or breast feeding.
18. Patients with uncontrolled anemia (Hb<10 g/dL in women; <12 g/dL in men).
19. Severe malnutrition (serum albumin<2.0 with a normal CRP).
20. At the time of placement of the silver microparticle-containing film Advanced Wound Care product or standard-of-care dressing, the subject has:
   i. Overt signs of wound infection as evidenced by, for example, purulence, erythema, cellulitis, excessively high temperatures in/around the ulcer, atypical smell, and/or excessive pain in/around the ulcer (infection may be treated and subject reconsidered for study participation).
   ii. Osteomyelitis, with necrotic soft bone. (x-ray to be obtained if in the opinion of the Investigator additional confirmation of diagnosis required)
   iii. Wounds healed by >20% in area during the 2-week, active run-in period
   iv. Use of hyperbaric oxygen, and active dressings that include growth factors, engineered tissues, or skin substitutes (e.g., Regranex, Dermagraft, Apligraf, etc.) within 30 days.
21. Wounds>10 cm² in surface area or >5 mm deep after sharp debridement.
22. Clinical suspicion of skin cancer at or near the ulcer location which has not been ruled out by biopsy.

Excluded Prior or Concomitant Medications
1. High doses of corticosteroids (i.e., doses≥1.5 mg/kg/day of prednisone or equivalent) within 90 days before study enrollment.
2. Chemotherapy or radiation therapy within 90 days before study enrollment.
3. Administration of immunoglobulin or granulocyte colony stimulating factor (G-CSF) within 90 days before study enrollment.

Treatment

Treatment is with a weekly application of the silver microparticle-containing film. At the baseline visit, necrotic tissue is removed through debridement using a sharp blade, scissors, curette or Versajet system. The silver microparticle-containing film will then be applied topically to cover the entire surface area of the wound followed by placement of a dressing for 7±2 days. Regularly scheduled dressing changes is done at the weekly (±2 days) study visits through Week 12 (i.e., no home dressing changes by the subject or caregiver) with the exception of the first scheduled dressing change which will occur on Day 2 (±1 day). After Week 12, dressing materials, frequency of dressing changes, and home dressing changes are at the Investigator's discretion for unhealed ulcers. The patient's diabetic foot ulcer is monitored on a weekly basis, including measurement of size and volume, along with assessment of ulcer deterioration, pain or signs of infection. Additional wound debridement is performed at subsequent clinic visits to remove any necrotic or unwanted tissue at the discretion of the Investigators.

Safety Evaluations

Day 2±1: Patients are evaluated within 2-3 days of the first application to assess local tolerability via increased local pain, edema, erythema, drainage, odor or signs of allergy (e.g., dermatitis).

Weeks 1-12: Investigators will look for evidence of infection in the diabetic foot ulcer. Adverse outcomes include increased redness, swelling, pain, odor or drainage. The Investigators are knowledgeable about the signs and symptoms of infections. If clinical signs of infection are present, then a wound culture is obtained via curettage at the ulcer base after aggressive sharp debridement. Appropriate systemic antibiotic treatment are administered until the infection resolves. Microbiology confirmation and choice of antibiotic therapy are recorded.

Weeks 1-12: Investigators will monitor for any deterioration of the ulcer, e.g., increased pain or drainage or increasing size. If there is evidence of deterioration of the ulcer, the silver microparticle-containing film treatments is discontinued, the patient will receive standard-of-care treatment and be followed for the duration of the study period. Swab cultures of unhealed wounds are obtained weekly and assayed for the presence of bacteria containing silver resistance genes.

Weeks 13-24: Patients are evaluated 12 (±2) weeks following discontinuation of silver microparticle-containing film treatment to determine the healing status of the DFU and presence of any long-term safety concerns.

While the invention has been particularly shown and described with reference to a preferred embodiment and various alternate embodiments, it is understood by persons skilled in the relevant art that various changes in form and details can be made therein without departing from the spirit and scope of the invention.

Statistical Methods

Continuous endpoints of pain, quality of life and ulcer size is compared by the Mann-Whitney U test. The Kaplan-Meier survival analysis is used to measure time to healing, and healing rate is measured using the log-rank χ2 test. Descriptive statistics, including mean, median, standard deviation, frequency and percentage are used to describe numerical data. Demographic variables, diabetes type, wound size, time to complete healing for wounds that healed and change in wound size from baseline to most recent follow-up evaluation for ulcers that did not heal are compared between the study groups. Pearson's chi-square analysis is used to compare categorical information. For continuous variables that are normally distributed, one-way analysis of variance is used to determine statistical significance. Using time to wound healing as the survival event, Kaplan-Meier survivorship analysis (product limit plot) is used to assess wound healing between treatment groups. Wounds are censored if they fail to heal by the 12-week endpoint or at the latest available clinical assessment or if the patient withdraws from the study. Wound measurements obtained at the latest follow-up evaluation is used for analysis. The log-rank test is used to identify significant differences between survival curves. Cox proportional hazards model analysis is conducted to determine the relationship between wound healing and the following covariates: ulcer size at presentation; index ulcer duration; patient age; body mass index; subject global assessment (SGA) of nutritional status; Site, Ischemia, Neuropathy, Bacterial Infection, Area and Depth (SINBAD) DFU classification score; and diabetes type. In addition, hazard ratio estimation is performed to evaluate the association between time to wound healing and the significant covariates. Statistical differences are considered significant when the P<0.05 with a power of at least 0.80. Ninety percent confidence intervals are used throughout the statistical analysis.

Data and Safety Monitoring Plan

Review of Safety Information. Vitruvian and an ERC will review safety data on an ongoing basis. The ERC is composed of specialists in diabetes (Robert Rushakoff, MD, Professor, UCSF), vascular surgery (Joseph H. Rapp, MD, Professor Emeritus, UCSF), and podiatric medicine (Chia-Ding (JD) Shih, DPM, MPH, MA, California School of Podiatric Medicine). The ERC will prepare study data reports for review by Vitruvian and inclusion in the final study report.

Study Stopping Rules. Enrollment and initiation of study treatment may be suspended at any time if any of the ERC reviews conclude that there are significant safety concerns. If the trial is put on hold, all screening, enrollment, and initiation of any new study subjects will cease pending Vitruvian's review.

The trial is placed on hold based on the occurrence of the following SAEs: Death or Serious Infections, defined as any infection Grade≥3 (as defined below). Vitruvian shall an evaluation of any unanticipated adverse effects and notify the ERC review safety data once every 3 months during planned Safety Data Review Meetings. Data for the planned safety reviews will include, at a minimum, a listing of all reported AEs and SAEs. In addition, the ERC may be called upon for ad hoc reviews. Vitruvian will promptly (within 5 business days) conduct.

Adverse effects are graded by Vitruvian and the attribution will ultimately be determined by the ERC.

Grading, Reporting and Attribution of Adverse Events. Grading Criteria. The study site will grade the severity of AEs experienced by study subjects according to the criteria set forth in the National Cancer Institute's Common Terminology Criteria for Adverse Events (NCICTCAE—version 4.0) for all AEs with the exception of infection. This document provides a common language to describe levels of severity, to analyze and interpret data, and to articulate the clinical significance of all AEs.

Adverse events are graded on a scale from 1 to 5 according to the following standards in the NCI-CTCAE manual:
Grade 1=mild adverse event.
Grade 2=moderate adverse event.
Grade 3=severe and undesirable adverse event.
Grade 4=life-threatening or disabling adverse event.
Grade 5=death.

Events Grade 2 or higher is recorded on the appropriate AE case report form for this study.

For any AE of infection, the following grading system is used for study participants:
Grade 1 infection=asymptomatic; clinical or diagnostic observation only; intervention with oral antimicrobial agents only; no invasive intervention required
Grade 2 infection=symptomatic; intervention with intravenous antimicrobial agents; invasive intervention may be required
Grade 3 infection=any infection associated with hemodynamic compromise requiring vasopressors, necessitating intensive care unit level of care or involving a necrotizing soft tissue infection
Grade 4 infection=life-threatening infection
Grade 5 infection=death resulting from infection

Reporting of Adverse Events. The Investigators will report all SAEs, regardless of relationship or expectedness within 24 hours of discovering the event to Vitruvian and IRB with all IND Safety Reports directed to the FDA. For SAEs, all requested information on the AE/SAE form is provided.

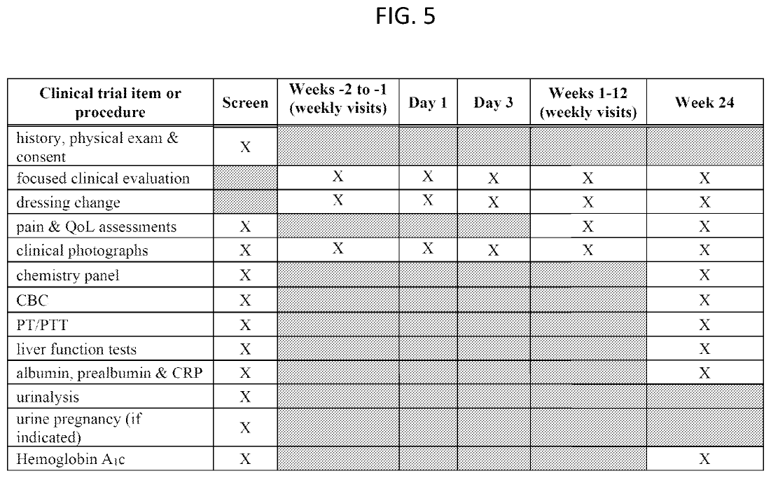

FIG. 5 provides a schedule of events for the proposed clinical trial.

Attribution Definitions. The relationship, or attribution, of an AE to the study regimen will initially be determined by the Investigator and recorded on the appropriate AE/SAE form. Final determination of attribution for safety reporting is determined by Vitruvian and the ERC. The relationship of an AE to study is determined as Unrelated, Possible or Definite via NCI-CTCAE definitions.

All references, issued patents and patent applications cited within the body of the instant specification are hereby incorporated by reference in their entirety, for all purposes.

1. A dried film, comprising: fibrin, fibrinogen, or combinations thereof and silver microparticles, wherein the film contains about 0.01 International Unit (IU) or less of thrombin per cm² of film; and wherein the film is stable at room temperature for at least 18 months.

2. The dried film of claim 1, wherein the silver microparticles are present at a concentration of 1-50 mg silver/cm² of film.

3. The dried film of claim 2, wherein the silver microparticles are present at a concentration of 2.5-25 mg silver microparticles/cm² of film.

4. The dried film of any one of claims 1-3, wherein the silver microparticles have a mean diameter ranging from about 2 μm to 1,000 μm.

5. The dried film of claim 4, wherein the silver microparticles have a mean diameter of about 15 μm.

6. The dried film of any one of the above claims, wherein the fibrin or fibrinogen is present as a component of whole blood or whole plasma.

7. The dried film of any one of the above claims, wherein the fibrin, fibrinogen or combinations thereof are present at an amount of 0.5 to 20.0 mg/cm² of film.

8. The dried film of claim 7, wherein the fibrinogen is present at an amount of 2.5 to 4.0 mg/cm² of film.

9. The dried film of any one of the above claims, wherein the film does not contain thrombin.

10. The dried film of any one of the above claims, wherein the film releases between 0.02 and 5.0 ppm silver ions when applied to a subject.

11. The dried film of any one of the above claims, wherein the film has a moisture content of between about 0.5 to 2.0 mg/cm²

12. The dried film of claim 11, wherein the film has a moisture content of about 0.9 mg/cm².

13. The dried film of any one of the above claims, wherein the film comprises silver microparticles on the surface of the film.

14. The dried film of any one of the above claims, wherein the silver microparticles on the surface of the film are abrasion resistant.

15. The dried film of claim 14, wherein the abrasion resistance is determined by brushing.

16. The dried film of claim 14 or 15, wherein the silver microparticles on the surface of the film are abrasion resistant to at least 100 brushing assay repeats.

17. The dried film of any one of the above claims, wherein the film has a fold number of at least 2, at least 3, at least 4, or at least 5.

18. The dried film of claim 17, wherein the fold number is determined by folding the film until the film breaks or ruptures.

19. The dried film of any one of the above claims, wherein the film has a fold endurance of at least 5, 10, 20, 30, 40, 50, 60, 70, 80, 90, 100, 500, or 1000.

20. The dried film of claim 19, wherein the fold endurance is determined by folding and unfolding the film on the same crease line until the film breaks or ruptures.

21. The dried film of any one of the above claims, wherein the film has a burst pressure of about 50 to 1000 mm.

22. The dried film of claim 21, wherein the film has a burst pressure of at least 800 mm Hg.

23. The dried film of any one of the above claims, wherein the film is stable at room temperature for at least 15 months.

24. The dried film of claim 23, wherein the stability is determined by burst pressure and fold endurance.

25. The dried film of claim 23 or 24, wherein the film has a fold number of at least 5 after storage at room temperature for 15 months.

26. The dried film of claim 23 or 24, wherein the film has a fold endurance of at least 100 after storage at room temperature for 15 months.

27. The dried film of claim 23 or 24, wherein the film has a burst pressure of at least 800 after storage at room temperature for 15 months.

28. The dried film of any one of the above claims, further comprising an adhesive coating.

29. The dried film of claim 28, wherein the adhesive coating is oxidized regenerated cellulose.

30. The dried film of claim 28 or 29, wherein the adhesive coating is present on the film at an amount of 5%-25% by weight of film.

31. The dried film of any one of the above claims, wherein the film is sterile.

32. The dried film of claim 31, wherein the film is sterilized by gamma irradiation.

33. The dried film of any one of the above claims, further comprising calcium.

34. The dried film of claim 33, wherein the calcium is present in the film at a concentration of 0.00005-0.20 mg/cm² of film.

35. The dried film of any one of the above claims, wherein the film has a thickness of between 0.1 mm and 1 mm.

36. The dried film of claim 35, wherein the film has a thickness of about 100 μm, 150 μm, or 200 μm.

37. A method of treating a wound in a subject, comprising application of an effective amount of the dried film of any one of claims 1-36 to the wound.

38. The method of claim 37, wherein the dried film is applied to the wound less than 15 minutes after opening a storage container comprising the dried film.

39. The method of claim 37, wherein the dried film is applied to the wound between 1 and 15 minutes after opening the storage container comprising the dried film.

40. The method of any one of claims 37-39, wherein the wound is an acute wound, a chronic wound, a non-healing wound, a diabetic foot ulcer, a venous leg ulcer, a pressure ulcer, a surgical wound, an arterial wound, a traumatic wound, or a skin disorder defined by ICD-9 code.

41. The method of claim 40, wherein the wound is a non-healing wound.

42. The method of claim 41, wherein the wound is selected from the group consisting of decreased primary post-surgical adhesion formation, decreased post-surgical adhesion reformation, a wound from cellular and tissue engraftment, and a burn wound.

43. The method of any one of claims 37-42, wherein the dried film degrades in less than 28 days after the application of the film to the wound of the subject.

44. A method of manufacturing a dried film for treating wounds in a subject, comprising:
obtaining human plasma;
placing the plasma into a mold;
polymerizing the plasma to form a gel by adding a polymerizing agent to the plasma;
adding silver microparticles to the gel to generate a silver microparticle-containing plasma gel;
removing fluid from the polymerized gel to generate a film;
drying the film; and
gamma sterilizing the film.

45. The method of claim 44, wherein the removal of fluid is performed by applying vacuum pressure to the gel at a pressure of about 1.25 Torr to 125 Torr.

46. The method of any one of claims 44-45, wherein the polymerizing agent is CaCl₂ or thrombin.

47. The method of claim 46, wherein the polymerizing agent is CaCl₂.

48. The method of claim 46 or 47, wherein between 0.5-1 mg/ml CaCl₂ is added to the plasma.

49. The method of claim 48, wherein about 0.86 mg/ml CaCl₂ is added to the plasma.

50. The method of any one of claims 44-49, further comprising incubating the film with a glycerin solution prior to drying the film.

51. The method of claim 50, wherein the glycerin solution comprises 2% glycerin.

52. The method of any one of claims 44-51, wherein the silver microparticles are added to the plasma.

53. The method of any one of claims 44-51, wherein the silver microparticles are added before polymerization of the gel.

54. The method of any one of claims 44-53, wherein the silver microparticles are added by mixing the silver microparticles with the plasma.

55. The method of any one of claims 44-51, wherein the silver microparticles are added after polymerization of the gel.

56. The method of any one of claims 44-51, wherein the silver microparticles are added before removal of the fluid from the gel.

57. The method of any one of claims 44-51, wherein the silver microparticles are added after removal of the fluid from the gel.

58. The method of any one of claims 44-51, wherein the silver microparticles are added after the glycerin solution incubation.

59. The method of any one of claims 44-51 and 55-58, wherein the silver microparticles are added by applying a spray coating comprising silver microparticles on the polymerized gel.

60. The method of any one of claims 44-59, wherein the silver microparticles are present at a concentration of 1-50 mg silver/cm² of film.

61. The method of claim 60, wherein the silver microparticles are present at a concentration of 2.5-25 mg silver/cm² of film.

62. The method of any one of claims 44-61, wherein the silver microparticles have a mean diameter ranging from 2 μm to 1,000 μm.

63. The method of claim 62, wherein the silver microparticles have a mean diameter of about 15 μm.

64. The method of any one of claims 44-63, wherein the film further comprises a phospholipid.

65. The method of claim 64, wherein the phospholipid is phosphatidylserine or phosphatidylcholine.

66. The method of any one of claims 44-65, wherein the calcium is present in the film at a concentration of 0.00005-0.20 mg/cm² of film.

67. The method of any one of claims 44-66, wherein the film comprises less than about 0.0999 IU thrombin per cm² of film.

68. The method of any one of claims 44-62, further comprising incubating the plasma at 37° C. after placing the plasma into the mold.

69. The method of claim 69, wherein the plasma is incubated at 37° C. for 5 to 30 min.

70. The method of claim 69, wherein the plasma is incubated at 37° C. for 15 min.

71. The method of any one of claims 44-62, further comprising incubating the gel at 37° C. after incubation with the glycerin solution.

72. The method of claim 71, wherein the gel is incubated at 37° C. for 5 to 60 min.

73. The method of claim 71, wherein the gel is incubated at 37° C. for 5 min.

74. The method of any one of claims 44-73, wherein the film is dried at a temperature of 42° C. to 50° C.

75. The method of any one of claims 44-74, wherein the film is dried for 30 min to 120 min.

76. The method of any one of claims 44-75, wherein the film is dried in a convection oven.

77. The method of any one of claims 44-76, wherein the film has a moisture content of between about 0.5 to 2.0 mg/cm²

78. The method of of claim 77, wherein the film has a moisture content of about 0.9 mg/cm².

79. The method of any one of claims 44-78, further comprising adding an adhesive coating to the film.

80. The method of claim 79, wherein the adhesive coating is oxidized regenerated cellulose.

81. The dried film of one of claim 79 or 80, wherein the adhesive coating is present on the film at an amount of 5%-25% by weight.

82. The method of any one of claims 44-81, further comprising virally inactivating the human plasma.

83. The method of claim 82, wherein the viral inactivation comprises solvent/detergent and/or nanofiltration.

84. The method of claim 83, wherein the solvent/detergent is Triton X-100.

85. The method of any one of claims 44-84, wherein the film has a thickness of about between 0.1 mm and 1 mm.

86. A method of preventing a wound in a subject, comprising application of an effective amount of the dried film of any one of claims 1-36 to an area that is susceptible to forming a wound in the subject.

87. The method of claim 86, wherein the dried film is applied to the area that is susceptible to forming a wound less than 15 minutes after opening a storage container comprising the dried film.

88. The method of claim 87, wherein the dried film is applied to the area that is susceptible to forming a wound between 1 and 15 minutes after opening the storage container comprising the dried film.

89. The method of any one of claims 86-88, wherein the wound is an acute wound, a chronic wound, a non-healing wound, a diabetic foot ulcer, a venous leg ulcer, a pressure ulcer, a surgical wound, an arterial wound, a traumatic wound, or a skin disorder defined by ICD-9 code.

90. The method of claim 89, wherein the wound is a non-healing wound.

91. The method of any one of claims 86-88, wherein the wound is a hernia.

92. The method of any one of claims 86-91, wherein the dried film degrades in less than 28 days after the application of the film to the area susceptible to forming a wound in the subject.
